(19) United States
(12) Patent Application Publication
(10) Pub. No.: US 2017/0145374 A1
(43) Pub. Date: May 25, 2017

(54) METHOD AND SYSTEM FOR CONTROLLING THE CONVERSION OF LIGNOCELLULOSIC MATERIALS

(71) Applicant: Stellenbosch University, Stellenbosch (ZA)

(72) Inventors: Willem Heber Van Zyl, Stellenbosch (ZA); Josebus Maree Van Zyl, Stellenbosch (ZA); Thomas Michael Harms, Stellenbosch (ZA); Eugene Van Rensburg, Tyger Waterfront (ZA); Lee Rybick Lynd, Meriden, NH (US)

(21) Appl. No.: 15/422,056

(22) Filed: Feb. 1, 2017

Related U.S. Application Data
(63) Continuation-in-part of application No. 13/808,592, filed on Apr. 3, 2013, now abandoned.

(30) Foreign Application Priority Data
Jul. 9, 2010 (ZA) 2010/04837

(51) Int. Cl. C12M 1/36; G05B 15/02; C12M 1/34

(52) U.S. Cl. CPC C12M 41/48; C12M 41/42; C12M 41/12; C12M 41/26; C12M 41/40; G05B 15/02

(57) ABSTRACT

The invention provides a method and system for controlling the conversion of crystalline insoluble cellulose to an organic product in a bioreactor containing crystalline insoluble cellulose and a culture medium. A processor of a computing device receives an input from a sensor in the bioreactor. The input may be a measurement of one or more of concentration, temperature, pH and pressure. The processor calculates conversion of cellulose using the input to provide a total calculated organic product in the bioreactor. The processor receives a further input from a sensor in the bioreactor of the total actual organic product and compares the total calculated organic product and the total actual organic product. The processor then transmits an instruction to an agitator associated with the bioreactor to control agitation of the content of the bioreactor if the total actual organic product is outside a predetermined range of the total calculated organic product.

### CROSS REFERENCE TO RELATED APPLICATION

For prosecution in the United States, this application is a Continuation-In-Part Application of Non-Provisional U.S. patent application Ser. No. 13/808,592 entitled “Systems for Modelling the Conversion of Lignocellulosic Materials” filed on Apr. 3, 2013, which claims priority to International Application No. PCT/162011/001590 of the same title, which was filed Jul. 8, 2011, and also claims priority to South African Application No. 2010/04837 of the same title, which was filed Jul. 9, 2010, all of which are entirely incorporated by reference herein.

### FIELD OF THE INVENTION

This invention relates to a method and system for controlling the conversion of lignocellulosic materials. More particularly, the invention relates to a method and system for controlling the conversion of crystalline cellulose hydrolysis through the activity of cellulase enzymes to release organic products.

### BACKGROUND TO THE INVENTION

Approximately 1.3×10¹⁰ metric tons (dry weight) of terrestrial plants are produced annually on a worldwide basis. Plant biomass consists of about 40% to 55% cellulose, 25% to 50% hemicellulose and 10% to 40% lignin, depending on whether the source is hardwood, softwood, or grasses. Cellulose is the major polysaccharide present, and is a water-insoluble glucan polymer that contains the major fraction of the fermentable sugar glucose.

Native cellulose consists of amorphous and crystalline regions and it is predominantly the former region that is prone to enzymatic attack. The major types of enzymatic activities required for native cellulose degradation are: endoglucanases, exoglucanases, cellobiohydrolases and β-glucosidases.

Endoglucanases randomly hydrolyses the cellulose polysaccharide chains in amorphous regions, generating oligosaccharides of varying lengths. Exoglucanases act in a processive manner on the reducing or non-reducing ends of these chains, liberating either glucose (glucanohydrolases) or cellobiose (cellobiohydrolase) as the major products. Exoglucanases can also act on microcrystalline cellulose, presumably peeling cellulose chains from the microcrystalline structure. β-Glucosidase enzymes hydrolyse soluble cellobiose to glucose units.

A variety of plant biomass resources are available as lignocellulosic feedstocks for the production of biofuels, notably bioethanol. The major sources are (i) wood residues from paper mills, sawmills and furniture manufacturing, (ii) municipal solid wastes, (iii) agricultural residues and (iv) energy crops. Pre-conversion of particularly the cellulosic fraction in these biomass resources (using either physical, chemical or enzymatic processes) to fermentable sugars (glucose and cellobiose) would enable their fermentation to bioethanol, provided the necessary fermentative micro-organisms with the ability to utilize these sugars are present.

Saccharomyces cerevisiae (Bakers' yeast) remains the preferred micro-organism for the production of ethanol. Attributes in favour of the use of this microbe include (i) high ethanol productivity approaching the theoretical ethanol yield (0.51 g ethanol produced/g glucose used), (ii) high osmo- and ethanol tolerance, (iii) natural robustness in industrial processes, (iv) being generally regarded as safe due to its long association with wine and bread making, and beer brewing. The major shortcoming of S. cerevisiae lies in its inability to utilize complex polysaccharides such as cellulose and the associated break-down products cellobiose and other cellodextrins. Therefore enzymes are added to break these complex polysaccharides down to simple sugars such as glucose which are easily fermented allowing for the simultaneous saccharification and fermentation (SSF) of the cellulose.

In an attempt to understand and predict SSF of cellulose, various numerical models have been proposed to describe the complex enzyme kinetics responsible for the hydrolysis of cellulose to sugar (Converse et al. 1988, Gusakov and Sinitsyn 1985, Scheiding et al. 1984, Caminal et al. 1985, Converse and Optekar 1993). However, limited literature exists on modelling complete SSF of cellulosic materials incorporating a fermentative yeast and exogenuously added cellulolytic enzymes.

South et al. (1995) proposed a model for the SSF of two pretreated hardwoods, namely birch and poplar. He assumed a Langmuir adsorption-type behaviour for the substrate-enzyme interactions and proposed a diminishing substrate conversion rate of the form

$${r}_{c} = ( {k ( 1 - x )}^{n} + c ) \times \frac{EC}{1 + {σ}_{c}}$$

as a function of conversion (x) and enzyme occupied active sites (EC) where k, n and c are empirical constants and σ_{c} the adsorption capacity of enzyme to the substrate. Shao et al. (2008) and Zhang et al. (2009) proposed similar models for paper sludge using dynamic adsorption. Parameters for adsorption and substrate conversion rates for these models were determined empirically from experimental measurements. The remaining rate equations and parameters describing the conversion of cellobiose to glucose and subsequent fermentation of glucose to ethanol were obtained from literature.

These models are, however, not very accurate, particularly as far as other heterogeneous/particulate cellulose sources are concerned. Also, being empirical models their usefulness tends to be limited, especially when scaling up reactions to commercial plant size.

Another factor which affects the scale up of chemical processes is the mixing conditions under which they occur. Most chemical reactor designs are based on the assumption that all components in the reactors are perfectly mixed. However in biological systems, the amount of mixing is limited by secondary conditions such as shear rate which could potentially be fatal to the organisms involved. Thus there exists a risk of incomplete mixing, which may result in the settling of particles out of suspension significantly reducing the efficiency and thus performance of these systems.

The present invention aims to alleviate these and other problems, at least to some extent.

The preceding discussion of the background to the invention is intended only to facilitate an understanding of the present invention. It should be appreciated that the discussion is not an acknowledgment or admission that any of the material referred to was part of the common general knowledge in the art as at the priority date of the application.

### SUMMARY OF THE INVENTION

In accordance with the invention, there is provided a computer-implemented method for controlling the conversion of crystalline insoluble cellulose to an organic product in a bioreactor containing crystalline insoluble cellulose and a culture medium, the method conducted at a processor of a computing device associated with the bioreactor and comprising
receiving an input from a sensor in the bioreactor, wherein the input is measurements of one or more of concentration, temperature, pH and pressure,
calculating conversion of cellulose using the input to provide a total calculated organic product in the bioreactor by solving the following equations:

$$\begin{matrix}\frac{d [ EC ]}{d t} = \frac{d [ C ]}{d t} ( 1 + {σ}_{e} ) + {k}_{fc} [ {E}_{f} ] [ {C}_{f} ] ( 1 + {σ}_{e} ) - \frac{{k}_{fc}}{K} [ EC ] & ( 1 ) \\ [ {E}_{f} ] = [ {E}_{T} ] - \frac{[ EC ] \times σ}{( 1 + σ )} & ( 2 ) \\ [ {C}_{f} ] = [ {C}_{T} ] - \frac{[ EC ]}{( 1 + σ )} & ( 3 ) \\ \frac{d [ C ]}{d t} = - k ( \frac{[ EC ]}{1 + σ} ) \times ( \frac{{K}_{C\_Cb}}{[ Cb ] + {K}_{C\_Cb}} ) \times ( \frac{{K}_{C\_Op}}{[ Op ] + {K}_{C\_Op}} ) & ( 4 ) \\ \frac{d [ Cb ]}{d t} = {K}_{f 1} \frac{d [ C ]}{d t} - \frac{{K}_{Cb} [ Cb ] [ B ]}{{K}_{m} \times ( ( 1 + \frac{[ G ]}{{K}_{Cb\_G}} ) + [ Cb ] )} & ( 5 ) \\ \frac{d [ G ]}{d t} = ( {K}_{f 1} \frac{d [ C ]}{d t} - \frac{d [ Cb ]}{d t} ) {K}_{f 2} - \frac{1}{{Y}_{X\_G}} \frac{d [ X ]}{d t} & ( 6 ) \\ \frac{d [ X ]}{d t} = \frac{{μ}_{max} [ X ] [ G ]}{[ G ] + {K}_{G}} \times ( 1 - \frac{[ Op ]}{{K}_{X\_Op}} ) & ( 7 ) \\ \frac{d [ Op ]}{d t} = ( \frac{{Y}_{Op\_G}}{{Y}_{X\_G}} ) \times \frac{d [ X ]}{d t} & ( 8 )\end{matrix}$$

where:
   K_{C}_{_}_{Cb}=Inhibition constant of cellobiose on cellulose conversion [g/L]
   K_{C}_{_}_{Op}=Inhibition constant of organic product on cellulose conversion [g/L]
   K_{Cb}=Rate constant for hydrolysis of cellobiose to glucose [g/L]
   K_{Cb}_{_}_{G}=Inhibition of hydrolysis of cellobiose by glucose [g/L]
   K=Equilibrium constant of enzyme [L/g]
   k=Hydrolysis rate constant of enzyme [h⁻¹]
   k_{fc}=Enzyme adsorption constant to cellulose [h⁻¹]
   K_{G}=Monod constant [g/L]
   Kₘ=Michaelis constant of enzyme for cellobiose [g/L]
   K_{X}_{_}_{Op}=Inhibition of cell growth by organic product [g/L]
   Y_{Op}_{_}_{G}=Yield of organic product cells per gram of glucose
   Y_{X}_{_}_{G}=Yield of organism cells per gram of glucose
   μₘₐₓ=Maximum growth rate of organism cells [h⁻¹]
   σₑ=Maximum bonding capacity of enzyme [dimensionless]
receiving a further input from a sensor in the bioreactor of the total actual organic product,
comparing the total calculated organic product and the total actual organic product, and,
transmitting an instruction to an agitator associated with the bioreactor to control agitation of the content of the bioreactor if the total actual organic product is outside a predetermined range of the total calculated organic product.

Further features of the invention provide for the method to include the processor receiving inputs from a plurality of sensors; for the inputs to be measurements of concentration and for the concentration to be enzyme loading concentration, cellulose concentration and organism concentration.

A further feature of the invention provides for the method to include the processor solving equations (1) to (8) iteratively.

A further feature of the invention provides for the method to include the processor receiving an additional input from a sensor in the bioreactor of a measurement relating to the rate of formation of enzyme-substrate complexes and calculating conversion of cellulose using this additional input to provide the total calculated organic product.

A further feature of the invention provides for the method to include the processor receiving an additional input from a sensor of a measurement relating to the oxygen supplied to the bioreactor, and calculating conversion of cellulose using this additional input to provide the total calculated organic product.

Further features of the invention provide for the method to include, if the total actual organic product is outside the predetermined range of the total calculated organic product, the processor transmitting an instruction to a heater associated with the bioreactor to control temperature in the bioreactor, transmitting an instruction to an inlet valve of the bioreactor to control addition of an acid or base to control pH in the bioreactor, and transmitting an instruction to a pressurizing component associated with the bioreactor to control pressure in the bioreactor.

Further features of the invention provide for the method to include, the processor transmitting instructions to control one or more of temperature, pH, and pressure within predetermined ranges.

Yet further features of the invention provide for the method to include the processor transmitting an instruction to an outlet valve of the bioreactor to cause purging of cellulose from the bioreactor if temperature is outside a predetermined temperature range.

Still further features of the invention provide for the method to further include receiving an input from a sensor relating to the degree of settling of particles in the medium in which the conversion of crystalline insoluble cellulose takes place, comparing the input to a predetermined settling threshold, and transmitting an instruction to an agitator associated with the bioreactor to control agitation of the content of the bioreactor if the comparison is outside the predetermined settling threshold.

Even further features of the invention provide for the organic product to be ethanol, glycerol, lactic acid, organic sugars, biomass, or lignin.

According to one aspect of the invention the organic product is ethanol and the culture medium includes Saccharomyces cerevisiae and wherein the method includes the processor
receiving the following inputs from one or more sensors in the bioreactor:
   Yeast cell concentration [g/L]—([X])
   Cellulose concentration [g/L]—([C])
   Cellobiose concentration [g/L]—([Cb])
   Exo-cellulase enzyme concentration [g/L]—([Eₑₓₒ])
   Endo-cellulase enzyme concentration [g/L]—([E_{endo}])
   β-Glucosidase concentration [g/L]—([B])
   Cellulose-enzyme complex concentration [g/L]—([EC]ₑₓₒ)
   Cellulose-enzyme complex concentration [g/L]—([EC]_{endo})
   Ethanol concentration [g/L]—([Eth])
   Glucose concentration [g/L]—([G])
calculating conversion of cellulose using these inputs to provide a total calculated ethanol in the bioreactor by solving the following equations:

$$\begin{matrix}\frac{d {[ EC ]}_{endo}}{d t} = \frac{d {[ C ]}_{endo}}{d t} \times ( 1 + {σ}_{endo} ) + {k}_{fc} [ {E}_{f , endo} ] [ {C}_{f , endo} ] ( 1 + {σ}_{endo} ) - {\frac{{k}_{fc}}{{K}_{endo}} [ EC ]}_{endo} & ( 9 ) \\ \frac{d {[ EC ]}_{exo}}{d t} = \frac{d {[ C ]}_{exo}}{d t} \times ( 1 + {σ}_{exo} ) + {k}_{fc} [ {E}_{f , exo} ] [ {C}_{f , exo} ] ( 1 + {σ}_{exo} ) - {\frac{{k}_{fc}}{{K}_{exo}} [ EC ]}_{exo} & ( 10 ) \\ [ {E}_{f} ] = [ {E}_{T} ] - \frac{[ EC ] \times σ}{( 1 + σ )} & ( 11 ) \\ [ {C}_{f} ] = [ {C}_{T} ] - \frac{[ EC ]}{( 1 + σ )} & ( 12 ) \\ \frac{d {[ C ]}_{endo}}{d t} = - {k}_{endo} \times \frac{{[ EC ]}_{endo}}{1 + {σ}_{endo}} \times ( \frac{{K}_{C\_Cb}}{[ Cb ] + {K}_{C\_Cb}} ) \times ( \frac{{K}_{C\_Eth}}{[ Eth ] + {K}_{C\_Eth}} ) & ( 13 ) \\ \frac{d {[ C ]}_{exo}}{d t} = tan h ( \frac{t}{τ} ) \times - {k}_{exo} \times \frac{{[ EC ]}_{exo}}{1 + {σ}_{exo}} \times ( \frac{{K}_{C\_Cb}}{[ Cb ] + {K}_{C\_Cb}} ) \times ( \frac{{K}_{C\_Eth}}{[ Eth ] + {K}_{C\_Eth}} ) & ( 14 ) \\ \frac{d [ Cb ]}{d t} = - \frac{342}{324} \times \frac{d [ C ]}{d t} - \frac{{K}_{Cb} [ Cb ] [ B ]}{{K}_{m} \times ( ( 1 + \frac{[ G ]}{{K}_{Cb\_G}} ) + [ Cb ] )} & ( 15 ) \\ \frac{d [ G ]}{d t} = ( - \frac{342}{324} \times \frac{d [ C ]}{d t} - \frac{d [ Cb ]}{d t} ) \times \frac{360}{342} - \frac{1}{{Y}_{X\_G}} \times \frac{d [ X ]}{d t} & ( 16 ) \\ \frac{d [ X ]}{d t} = \frac{{μ}_{max} [ X ] [ G ]}{[ G ] + {K}_{G}} \times ( 1 - \frac{[ Eth ]}{{K}_{X\_Eth}} ) & ( 17 ) \\ \frac{d [ Eth ]}{d t} = ( \frac{{Y}_{Eth\_G}}{{Y}_{X\_G}} ) \times \frac{d [ X ]}{d t} & ( 18 )\end{matrix}$$

where:
   K_{C}_{_}_{Cb}=Inhibition constant of cellobiose on cellulose conversion [g/L]
   K_{C}_{_}_{Eth}=Inhibition constant of ethanol on cellulose conversion [g/L]
   K_{Cb}=Rate constant for hydrolysis of cellobiose to glucose [g/L]
   K_{Cb}_{_}_{G}=Inhibition of hydrolysis of cellobiose by glucose [g/L]
   K_{endo}=Equilibrium constant for endoglucanase [L/g]
   k_{endo}=Hydrolysis rate constant of endoglucanase [h⁻¹]
   Kₑₓₒ=Equilibrium constant for exoglucanase [L/g]
   kₑₓₒ=Hydrolysis rate constant of exoglucanase [h⁻¹]
   k_{fc}=Enzyme adsorption constant to Avicel [h⁻¹]
   K_{G}=Monod constant [g/L]
   Kₘ=Michaelis constant of β-glucosidase for cellobiose [g/L]
   K_{X}_{_}_{Eth}=Inhibition of cell growth by ethanol [g/L]
   Y_{Eth}_{_}_{G}=Yield of ethanol cells per gram of glucose
   Y_{X}_{_}_{G}=Yield of yeast cells per gram of glucose
   μₘₐₓ=Maximum growth rate of yeast cells [h⁻¹]
   σ_{endo}=Endoglucanse enzyme capacity on Avicel [dimensionless]
   σₑₓₒ=Exoglucanase enzyme capacity on Avicel [dimensionless]
receiving a further input from a sensor in the bioreactor of the total actual ethanol,
comparing the total calculated ethanol and the total actual ethanol, and,
transmitting an instruction to an agitator associated with the bioreactor to control agitation of the content of the bioreactor if the total actual ethanol is outside a predetermined range of the total calculated ethanol.

A further feature of the invention provides for the method to include the processor solving equations (9) to (18) iteratively.

According to one aspect of the invention the organic product is glycerol and the culture medium includes Saccharomyces cerevisiae and wherein the method includes the processor
receiving the following inputs from one or more sensors in the bioreactor:
   Yeast cell concentration [g/L]—([X])
   Cellulose concentration [g/L]—([C])
   Cellobiose concentration [g/L]—([Cb])
   Exo-cellulase enzyme concentration [g/L]—([Eₑₓₒ])
   Endo-cellulase enzyme concentration [g/L]—([E_{endo}])
   β-Glucosidase concentration [g/L]—([B])
   Cellulose-enzyme complex concentration [g/L]—([EC]ₑₓₒ),
   Cellulose-enzyme complex concentration [g/L]—([EC]_{endo}),
   Glycerol concentration [g/L]—([Gly])
   Glucose concentration [g/L]—([G])
calculating conversion of cellulose using these inputs to provide a total calculated glycerol in the bioreactor by solving the following equations:

$$\begin{matrix}\frac{d {[ EC ]}_{endo}}{d t} = \frac{d {[ C ]}_{endo}}{d t} \times ( 1 + {σ}_{endo} ) + {k}_{fc} [ {E}_{f , endo} ] [ {C}_{f , endo} ] ( 1 + {σ}_{endo} ) - {\frac{{k}_{fc}}{{K}_{endo}} [ EC ]}_{endo} & ( 9 ) \\ \frac{d {[ EC ]}_{exo}}{d t} = \frac{d {[ C ]}_{exo}}{d t} \times ( 1 + {σ}_{exo} ) + {k}_{fc} [ {E}_{f , exo} ] [ {C}_{f , exo} ] ( 1 + {σ}_{exo} ) - {\frac{{k}_{fc}}{{K}_{exo}} [ EC ]}_{exo} & ( 10 ) \\ [ {E}_{f} ] = [ {E}_{T} ] - \frac{[ EC ] \times σ}{( 1 + σ )} & ( 11 ) \\ [ {C}_{f} ] = [ {C}_{T} ] - \frac{[ EC ]}{( 1 + σ )} & ( 12 ) \\ \frac{d {[ C ]}_{endo}}{d t} = - {k}_{endo} \times \frac{{[ EC ]}_{endo}}{1 + {σ}_{endo}} \times ( \frac{{K}_{C\_Cb}}{[ Cb ] + {K}_{C\_Cb}} ) \times ( \frac{{K}_{C\_Gly}}{[ Gly ] + {K}_{C\_Gly}} ) & ( 19 ) \\ \frac{d {[ C ]}_{exo}}{d t} = tan h ( \frac{t}{τ} ) \times - {k}_{exo} \times \frac{{[ EC ]}_{exo}}{1 + {σ}_{exo}} \times ( \frac{{K}_{C\_Cb}}{[ Cb ] + {K}_{C\_Cb}} ) \times ( \frac{{K}_{C\_Gly}}{[ Gly ] + {K}_{C\_Gly}} ) & ( 20 ) \\ \frac{d [ Cb ]}{d t} = - \frac{342}{324} \times \frac{d [ C ]}{d t} - \frac{{K}_{Cb} [ Cb ] [ B ]}{{K}_{m} \times ( ( 1 + \frac{[ G ]}{{K}_{Cb\_G}} ) + [ Cb ] )} & ( 15 ) \\ \frac{d [ G ]}{d t} = ( - \frac{342}{324} \times \frac{d [ C ]}{d t} - \frac{d [ Cb ]}{d t} ) \times \frac{360}{342} - \frac{1}{{Y}_{X\_G}} \times \frac{d [ X ]}{d t} & ( 16 ) \\ \frac{d [ X ]}{d t} = \frac{{μ}_{max} [ X ] [ G ]}{[ G ] + {K}_{G}} \times ( 1 - \frac{[ Gly ]}{{K}_{X\_Gly}} ) & ( 21 ) \\ \frac{d [ Gly ]}{d t} = ( \frac{{Y}_{Gly\_G}}{{Y}_{X\_G}} ) \times \frac{d [ X ]}{d t} & ( 22 )\end{matrix}$$

where:
   K_{C}_{_}_{Gly}=Inhibition constant of glycerol on cellulose conversion [g/L]
   K_{X}_{_}_{Gly}=Inhibition of cell growth by glycerol [g/L]
   Y_{Gly}_{_}_{G}=Yield of glycerol cells per gram of glucose
receiving a further input from a sensor in the bioreactor of the total actual glycerol,
comparing the total calculated glycerol and the total actual glycerol, and,
transmitting an instruction to an agitator associated with the bioreactor to control agitation of the content of the bioreactor if the total actual glycerol is outside a predetermined range of the total calculated glycerol.

A further feature of the invention provides for the method to include the processor solving equations (9) to (12), (15) to (16) and (19) to (22) iteratively.

The invention also provides a system for controlling the conversion of crystalline insoluble cellulose to an organic product in a bioreactor which can hold crystalline insoluble cellulose and a culture medium, the system comprising a computing device with memory for storing computer-readable program code and a processor for executing the computer-readable program code, wherein the processor is configured to interact with one or more sensors in the bioreactor, and an agitator associated with the bioreactor, and wherein the processor includes:
a receiving component for receiving an input from a sensor, wherein the input is measurements of one or more of concentration, temperature, pH and pressure,
a calculating component for calculating conversion of cellulose using the input to provide a total calculated organic product in the bioreactor by solving the following equations:

$$\begin{matrix}\frac{d [ EC ]}{d t} = \frac{d [ C ]}{d t} ( 1 + {σ}_{e} ) + {k}_{fc} [ {E}_{f} ] [ {C}_{f} ] ( 1 + {σ}_{e} ) - \frac{{k}_{fc}}{K} [ EC ] & ( 1 ) \\ [ {E}_{f} ] = [ {E}_{T} ] - \frac{[ EC ] \times σ}{( 1 + σ )} & ( 2 ) \\ [ {C}_{f} ] = [ {C}_{T} ] - \frac{[ EC ]}{( 1 + σ )} & ( 3 ) \\ \frac{d [ C ]}{d t} = - k ( \frac{[ EC ]}{1 + σ} ) \times ( \frac{{K}_{C\_Cb}}{[ Cb ] + {K}_{C\_Cb}} ) \times ( \frac{{K}_{C\_Op}}{[ Op ] + {K}_{C\_Op}} ) & ( 4 ) \\ \frac{d [ Cb ]}{d t} = {K}_{f 1} \frac{d [ C ]}{d t} - \frac{{K}_{Cb} [ Cb ] [ B ]}{{K}_{m} \times ( ( 1 + \frac{[ G ]}{{K}_{Cb\_G}} ) + [ Cb ] )} & ( 5 ) \\ \frac{d [ G ]}{d t} = ( {K}_{f 1} \frac{d [ C ]}{d t} - \frac{d [ Cb ]}{d t} ) {K}_{f 2} - \frac{1}{{Y}_{X\_G}} \frac{d [ X ]}{d t} & ( 6 ) \\ \frac{d [ X ]}{d t} = \frac{{μ}_{max} [ X ] [ G ]}{[ G ] + {K}_{G}} \times ( 1 - \frac{[ Op ]}{{K}_{X\_Op}} ) & ( 7 ) \\ \frac{d [ Op ]}{d t} = ( \frac{{Y}_{Op\_G}}{{Y}_{X\_G}} ) \times \frac{d [ X ]}{d t} & ( 8 )\end{matrix}$$

where:
   K_{C}_{_}_{Cb}=Inhibition constant of cellobiose on cellulose conversion [g/L]
   K_{C}_{_}_{Op}=Inhibition constant of organic product on cellulose conversion [g/L]
   K_{Cb}=Rate constant for hydrolysis of cellobiose to glucose [g/L]
   K_{Cb}_{_}_{G}=Inhibition of hydrolysis of cellobiose by glucose [g/L]
   K=Equilibrium constant of enzyme [L/g]
   k=Hydrolysis rate constant of enzyme [h⁻¹]
   k_{fc}=Enzyme adsorption constant to cellulose [h⁻¹]
   K_{G}=Monod constant [g/L]
   Kₘ=Michaelis constant of enzyme for cellobiose [g/L]
   K_{X}_{_}_{Op}=Inhibition of cell growth by organic product [g/L]
   Y_{Op}_{_}_{G}=Yield of organic product cells per gram of glucose
   Y_{X}_{_}_{G}=Yield of organism cells per gram of glucose
   μₘₐₓ=Maximum growth rate of organism cells [h⁻¹]
   σₑ=Maximum bonding capacity of enzyme [dimensionless]
the receiving component receiving a further input from a sensor in the bioreactor of the total actual organic product,
a comparing component for comparing the total calculated organic product and the total actual organic product, and,
an agitating component for transmitting an instruction to an agitator associated with the bioreactor to control agitation of the content of the bioreactor if the total actual organic product is outside a predetermined range of the total calculated organic product.

Further features of the invention provide for the receiving component of the processor to be configured to receive inputs from a plurality of sensors in the bioreactor; for the inputs to be measurements of concentration and for the concentration to be enzyme loading concentration, cellulose concentration and organism concentration.

Further features of the invention provide for the calculating component of the processor to be configured to solve equations (1) to (8) iteratively.

Further features of the invention provide for the receiving component of the processor to be configured to receive an additional input from a sensor in the bioreactor of a measurement relating to the rate of formation of enzyme-substrate complexes and for the calculating component of the processor to be configured to calculate conversion of cellulose using this additional input to provide the total calculated organic product.

Further features of the invention provide for the receiving component of the processor to be configured to receive an additional input from a sensor of a measurement relating to the oxygen supplied to the bioreactor and for the calculating component of the processor to be configured to calculate conversion of cellulose using this additional input to provide the total calculated organic product.

A further feature of the invention provides for the processor to include a temperature component for transmitting an instruction to a heater associated with the bioreactor to control temperature in the bioreactor if the total actual organic product is outside the predetermined range of the total calculated organic product and for transmitting an instruction to control the temperature in the bioreactor within a predetermined temperature range.

A further feature of the invention provides for the temperature component of the processor to be configured to transmit an instruction to an outlet valve of the bioreactor to cause purging of cellulose from the bioreactor if temperature is outside the predetermined temperature range.

A further feature of the invention provides for the processor to include a pH component for transmitting an instruction to an inlet valve of the bioreactor to control addition of an acid or base to control pH in the bioreactor if the total actual organic product is outside the predetermined range of the total calculated organic product and for transmitting an instruction to control the pH in the bioreactor within a predetermined pH range.

A further feature of the invention provides for the processor to include a pressure component for transmitting an instruction to a pressurizing component associated with the bioreactor to control pressure in the bioreactor if the total actual organic product is outside the predetermined range of the total calculated organic product and for transmitting an instruction to control the pressure in the bioreactor within a predetermined pressure range.

Further features of the invention provide for the receiving component of the processor to be configured to receive an input from a sensor relating to the degree of settling of particles in the medium in which the conversion of crystalline insoluble cellulose takes place, the comparing component of the processor to be configured to compare the input to a predetermined settling threshold, and for the agitating component of the processor to be configured to transmit an instruction to an agitator associated with the bioreactor to control agitation of the content of the bioreactor if the comparison is outside the predetermined settling threshold.

According to one aspect of the invention the organic product is ethanol and the culture medium includes Saccharomyces cerevisiae and wherein the system includes,
the receiving component receiving the following inputs from one or more sensors in the bioreactor:
   Yeast cell concentration [g/L]—([X])
   Cellulose concentration [g/L]—([C])
   Cellobiose concentration [g/L]—([Cb])
   Exo-cellulase enzyme concentration [g/L]—([Eₑₓₒ])
   Endo-cellulase enzyme concentration [g/L]—([E_{endo}])
   β-Glucosidase concentration [g/L]—([B])
   Cellulose-enzyme complex concentration [g/L]—([EC]ₑₓₒ),
   Cellulose-enzyme complex concentration [g/L]—([EC]_{endo}),
   Ethanol concentration [g/L]—([Eth])
   Glucose concentration [g/L]—([G]),
the calculating component calculating conversion of cellulose using these inputs to provide a total calculated ethanol in the bioreactor by solving the following equations:

$$\begin{matrix}\frac{d {[ EC ]}_{endo}}{d t} = \frac{d {[ C ]}_{endo}}{d t} \times ( 1 + {σ}_{endo} ) + {k}_{fc} [ {E}_{f , endo} ] [ {C}_{f , endo} ] ( 1 + {σ}_{endo} ) - {\frac{{k}_{fc}}{{K}_{endo}} [ EC ]}_{endo} & ( 9 ) \\ \frac{d {[ EC ]}_{exo}}{d t} = \frac{d {[ C ]}_{exo}}{d t} \times ( 1 + {σ}_{exo} ) + {k}_{fc} [ {E}_{f , exo} ] [ {C}_{f , exo} ] ( 1 + {σ}_{exo} ) - {\frac{{k}_{fc}}{{K}_{exo}} [ EC ]}_{exo} & ( 10 ) \\ [ {E}_{f} ] = [ {E}_{T} ] - \frac{[ EC ] \times σ}{( 1 + σ )} & ( 11 ) \\ [ {C}_{f} ] = [ {C}_{T} ] - \frac{[ EC ]}{( 1 + σ )} & ( 12 ) \\ \frac{d {[ C ]}_{endo}}{d t} = - {k}_{endo} \times \frac{{[ EC ]}_{endo}}{1 + {σ}_{endo}} \times ( \frac{{K}_{C\_Cb}}{[ Cb ] + {K}_{C\_Cb}} ) \times ( \frac{{K}_{C\_Eth}}{[ Eth ] + {K}_{C\_Eth}} ) & ( 13 ) \\ \frac{d {[ C ]}_{exo}}{d t} = tan h ( \frac{t}{τ} ) \times - {k}_{exo} \times \frac{{[ EC ]}_{exo}}{1 + {σ}_{exo}} \times ( \frac{{K}_{C\_Cb}}{[ Cb ] + {K}_{C\_Cb}} ) \times ( \frac{{K}_{C\_Eth}}{[ Eth ] + {K}_{C\_Eth}} ) & ( 14 ) \\ \frac{d [ Cb ]}{d t} = - \frac{342}{324} \times \frac{d [ C ]}{d t} - \frac{{K}_{Cb} [ Cb ] [ B ]}{{K}_{m} \times ( ( 1 + \frac{[ G ]}{{K}_{Cb\_G}} ) + [ Cb ] )} & ( 15 ) \\ \frac{d [ G ]}{d t} = ( - \frac{342}{324} \times \frac{d [ C ]}{d t} - \frac{d [ Cb ]}{d t} ) \times \frac{360}{342} - \frac{1}{{Y}_{X\_G}} \times \frac{d [ X ]}{d t} & ( 16 ) \\ \frac{d [ X ]}{d t} = \frac{{μ}_{max} [ X ] [ G ]}{[ G ] + {K}_{G}} \times ( 1 - \frac{[ Eth ]}{{K}_{X\_Eth}} ) & ( 17 ) \\ \frac{d [ Eth ]}{d t} = ( \frac{{Y}_{Eth\_G}}{{Y}_{X\_G}} ) \times \frac{d [ X ]}{d t} & ( 18 )\end{matrix}$$

where:
   K_{C}_{_}_{Cb}=Inhibition constant of cellobiose on cellulose conversion [g/L]
   K_{C}_{_}_{Eth}=Inhibition constant of ethanol on cellulose conversion [g/L]
   K_{Cb}=Rate constant for hydrolysis of cellobiose to glucose [g/L]
   K_{Cb}_{_}_{G}=Inhibition of hydrolysis of cellobiose by glucose [g/L]
   K_{endo}=Equilibrium constant for endoglucanase [L/g]
   k_{endo}=Hydrolysis rate constant of endoglucanase [h⁻¹]
   Kₑₓₒ=Equilibrium constant for exoglucanase [L/g]
   kₑₓₒ=Hydrolysis rate constant of exoglucanase [h⁻¹]
   k_{fc}=Enzyme adsorption constant to Avicel [h⁻¹]
   K_{G}=Monod constant [g/L]
   Kₘ=Michaelis constant of β-glucosidase for cellobiose [g/L]
   K_{X}_{_}_{Eth}=Inhibition of cell growth by ethanol [g/L]
   Y_{Eth}_{_}_{G}=Yield of ethanol cells per gram of glucose
   Y_{X}_{_}_{G}=Yield of yeast cells per gram of glucose
   μₘₐₓ=Maximum growth rate of yeast cells [h⁻¹]
   σ_{endo}=Endoglucanse enzyme capacity on Avicel [dimensionless]
   σₑₓₒ=Exoglucanase enzyme capacity on Avicel [dimensionless]
   τ=Time Constant [h]
the receiving component receiving a further input from a sensor in the bioreactor of the total actual ethanol,
the comparing component comparing the total calculated ethanol and the total actual ethanol, and,
the agitating component transmitting an instruction to an agitator associated with the bioreactor to control agitation of the content of the bioreactor if the total actual ethanol is outside a predetermined range of the total calculated ethanol.

Further features of the invention provide for the calculating component of the processor to be configured to solve equations (9) to (18) iteratively.

According to one aspect of the invention the organic product is glycerol and the culture medium includes Saccharomyces cerevisiae and wherein the system includes,
the receiving component receiving the following inputs from one or more sensors in the bioreactor:
   Yeast cell concentration [g/L]—([X])
   Cellulose concentration [g/L]—([C])
   Cellobiose concentration [g/L]—([Cb])
   Exo-cellulase enzyme concentration [g/L]—([Eₑₓₒ])
   Endo-cellulase enzyme concentration [g/L]—([E_{endo}])
   β-Glucosidase concentration [g/L]—([B])
   Cellulose-enzyme complex concentration [g/L]—([EC]ₑₓₒ)
   Cellulose-enzyme complex concentration [g/L]—([EC]_{endo})
   Glycerol concentration [g/L]—([Gly])
   Glucose concentration [g/L]—([G])
the calculating component calculating conversion of cellulose using these inputs to provide a total calculated glycerol in the bioreactor by solving the following equations:

$$\begin{matrix}\frac{d {[ EC ]}_{endo}}{d t} = \frac{d {[ C ]}_{endo}}{d t} \times ( 1 + {σ}_{endo} ) + {k}_{fc} [ {E}_{f , endo} ] [ {C}_{f , endo} ] ( 1 + {σ}_{endo} ) - {\frac{{k}_{fc}}{{K}_{endo}} [ EC ]}_{endo} & ( 9 ) \\ \frac{d {[ EC ]}_{exo}}{d t} = \frac{d {[ C ]}_{exo}}{d t} \times ( 1 + {σ}_{exo} ) + {k}_{fc} [ {E}_{f , exo} ] [ {C}_{f , exo} ] ( 1 + {σ}_{exo} ) - {\frac{{k}_{fc}}{{K}_{exo}} [ EC ]}_{exo} & ( 10 ) \\ [ {E}_{f} ] = [ {E}_{T} ] - \frac{[ EC ] \times σ}{( 1 + σ )} & ( 11 ) \\ [ {C}_{f} ] = [ {C}_{T} ] - \frac{[ EC ]}{( 1 + σ )} & ( 12 ) \\ \frac{d {[ C ]}_{endo}}{d t} = - {k}_{endo} \times \frac{{[ EC ]}_{endo}}{1 + {σ}_{endo}} \times ( \frac{{K}_{C\_Cb}}{[ Cb ] + {K}_{C\_Cb}} ) \times ( \frac{{K}_{C\_Gly}}{[ Gly ] + {K}_{C\_Gly}} ) & ( 19 ) \\ \frac{d {[ C ]}_{exo}}{d t} = tan h ( \frac{t}{τ} ) \times - {k}_{exo} \times \frac{{[ EC ]}_{exo}}{1 + {σ}_{exo}} \times ( \frac{{K}_{C\_Cb}}{[ Cb ] + {K}_{C\_Cb}} ) \times ( \frac{{K}_{C\_Gly}}{[ Gly ] + {K}_{C\_Gly}} ) & ( 20 ) \\ \frac{d [ Cb ]}{d t} = - \frac{342}{324} \times \frac{d [ C ]}{d t} - \frac{{K}_{Cb} [ Cb ] [ B ]}{{K}_{m} \times ( ( 1 + \frac{[ G ]}{{K}_{Cb\_G}} ) + [ Cb ] )} & ( 15 ) \\ \frac{d [ G ]}{d t} = ( - \frac{342}{324} \times \frac{d [ C ]}{d t} - \frac{d [ Cb ]}{d t} ) \times \frac{360}{342} - \frac{1}{{Y}_{X\_G}} \times \frac{d [ X ]}{d t} & ( 16 ) \\ \frac{d [ X ]}{d t} = \frac{{μ}_{max} [ X ] [ G ]}{[ G ] + {K}_{G}} \times ( 1 - \frac{[ Gly ]}{{K}_{X\_Gly}} ) & ( 21 ) \\ \frac{d [ Gly ]}{d t} = ( \frac{{Y}_{Gly\_G}}{{Y}_{X\_G}} ) \times \frac{d [ X ]}{d t} & ( 22 )\end{matrix}$$

where:
   K_{C}_{_}_{Gly}=Inhibition constant of glycerol on cellulose conversion [g/L]
   K_{X}_{_}_{Gly}=Inhibition of cell growth by glycerol [g/L]
   Y_{Gly}_{_}_{G}=Yield of glycerol cells per gram of glucose
the receiving component receiving a further input from a sensor in the bioreactor of the total actual glycerol,
the comparing component comparing the total calculated glycerol and the total actual glycerol, and,
the agitating component transmitting an instruction to an agitator associated with the bioreactor to control agitation of the content of the bioreactor if the total actual glycerol is outside a predetermined range of the total calculated glycerol.

Further features of the invention provide for the calculating component of the processor to be configured to solve equations (9) to (12), (15) to (16) and (19) to (22) iteratively.

The invention also provides a computer program product for controlling the conversion of crystalline insoluble cellulose to an organic product in a bioreactor containing crystalline insoluble cellulose and a culture medium, the computer program product comprising a computer-readable medium having stored computer-readable program code for performing the steps of
receiving an input from a sensor in the bioreactor, wherein the input is measurements of one or more of concentration, temperature, pH and pressure,
calculating conversion of cellulose using the input to provide a total calculated organic product in the bioreactor by solving the following equations:

$$\begin{matrix}\frac{d [ EC ]}{d t} = \frac{d [ C ]}{d t} ( 1 + {σ}_{e} ) + {k}_{fc} [ {E}_{f} ] [ {C}_{f} ] ( 1 + {σ}_{e} ) - \frac{{k}_{fc}}{K} [ EC ] & ( 1 ) \\ [ {E}_{f} ] = [ {E}_{T} ] - \frac{[ EC ] \times σ}{( 1 + σ )} & ( 2 ) \\ [ {C}_{f} ] = [ {C}_{T} ] - \frac{[ EC ]}{( 1 + σ )} & ( 3 ) \\ \frac{d [ C ]}{d t} = - k ( \frac{[ EC ]}{1 + σ} ) \times ( \frac{{K}_{C\_Cb}}{[ Cb ] + {K}_{C\_Cb}} ) \times ( \frac{{K}_{C\_Op}}{[ Op ] + {K}_{C\_Op}} ) & ( 4 ) \\ \frac{d [ Cb ]}{d t} = {K}_{f 1} \frac{d [ C ]}{d t} - \frac{{K}_{Cb} [ Cb ] [ B ]}{{K}_{m} \times ( ( 1 + \frac{[ G ]}{{K}_{Cb\_G}} ) + [ Cb ] )} & ( 5 ) \\ \frac{d [ G ]}{d t} = ( {K}_{f 1} \frac{d [ C ]}{d t} - \frac{d [ Cb ]}{d t} ) {K}_{f 2} - \frac{1}{{Y}_{X\_G}} \frac{d [ X ]}{d t} & ( 6 ) \\ \frac{d [ X ]}{d t} = \frac{{μ}_{max} [ X ] [ G ]}{[ G ] + {K}_{G}} \times ( 1 - \frac{[ Op ]}{{K}_{X\_Op}} ) & ( 7 ) \\ \frac{d [ Op ]}{d t} = ( \frac{{Y}_{Op\_G}}{{Y}_{X\_G}} ) \times \frac{d [ X ]}{d t} & ( 8 )\end{matrix}$$

where:
   K_{C}_{_}_{Cb}=Inhibition constant of cellobiose on cellulose conversion [g/L]
   K_{C}_{_}_{Op}=Inhibition constant of organic product on cellulose conversion [g/L]
   K_{Cb}=Rate constant for hydrolysis of cellobiose to glucose [g/L]
   K_{Cb}_{_}_{G}=Inhibition of hydrolysis of cellobiose by glucose [g/L]
   K=Equilibrium constant of enzyme [L/g]
   k=Hydrolysis rate constant of enzyme [h⁻¹]
   k_{fc}=Enzyme adsorption constant to cellulose [h⁻¹]
   K_{G}=Monod constant [g/L]
   Kₘ=Michaelis constant of enzyme for cellobiose [g/L]
   K_{X}_{_}_{Op}=Inhibition of cell growth by organic product [g/L]
   Y_{Op}_{_}_{G}=Yield of organic product cells per gram of glucose
   Y_{X}_{_}_{G}=Yield of organism cells per gram of glucose
   μₘₐₓ=Maximum growth rate of organism cells [h⁻¹]
   σₑ=Maximum bonding capacity of enzyme [dimensionless]
receiving a further input from a sensor in the bioreactor of the total actual organic product,
comparing the total calculated organic product and the total actual organic product, and,
transmitting an instruction to an agitator associated with the bioreactor to control agitation of the content of the bioreactor if the total actual organic product is outside a predetermined range of the total calculated organic product.

The computer-readable medium may be a non-transitory computer-readable medium and the computer-readable program code may be executable by a processor.

Embodiments of the invention will now be described, by way of example only, with reference to the accompanying drawings.

### BRIEF DESCRIPTION OF THE DRAWINGS

In the drawings:—

FIG. 1 is a schematic diagram of a system according to embodiments of the present invention;

FIG. 2 is a block diagram which illustrates controlling agitation of the content of the bioreactor according to an embodiment of the present invention;

FIG. 3 is a plot of concentration [g/L] (y-axis) in against time [h] (x-axis) for the experimental and calculated data for glucose, ethanol, glycerol and biomass;

FIG. 4 is a plot of protein concentration [g/L] (y-axis) in against time [h] (x-axis) for the calculated added and free enzymes in solution for endoglucanase and exoglucanase;

FIG. 5 is a plot of protein concentration [g/L] (y-axis) in against time [h] (x-axis) for the calculated and simulated adsorbed enzymes in solution for endoglucanase and exoglucanase;

FIG. 6 is a plot of concentration [g/L] (y-axis) in against time [h] (x-axis) for the experimental and simulated data for Avicel, glucose, ethanol, glycerol and biomass;

FIG. 7 is a plot showing the dynamic viscosity for Avicel particles in water with the error-bars representing the standard deviation of each measurement;

FIG. 8 is a block diagram which illustrates logical components of an exemplary computing device that may be used in embodiments of the disclosure; and,

FIG. 9 is a block diagram which illustrates an example of a computing device in which various aspects of the disclosure may be implemented.

### DETAILED DESCRIPTION WITH REFERENCE TO THE DRAWINGS

The invention provides a method and system for controlling the conversion of crystalline insoluble cellulose to an organic product in a bioreactor containing crystalline insoluble cellulose and a culture medium. A processor of a computing device receives an input from a sensor in the bioreactor. The input may be a measurement of one or more of concentration, temperature, pH and pressure. The processor calculates conversion of cellulose using the input to provide a total calculated organic product in the bioreactor.

The processor calculates conversion of cellulose by solving the following equations:

$$\begin{matrix}\frac{d [ E C ]}{d t} = \frac{d [ C ]}{d t} ( 1 + {σ}_{e} ) + {k}_{fc} [ {E}_{f} ] [ {C}_{f} ] ( 1 + {σ}_{e} ) - \frac{{k}_{fc}}{K} [ E C ] & ( 1 ) \\ [ {E}_{f} ] = [ {E}_{T} ] - \frac{[ E C ] \times σ}{( 1 + σ )} & ( 2 ) \\ [ {C}_{f} ] = [ {C}_{T} ] - \frac{[ E C ]}{( 1 + σ )} & ( 3 ) \\ \frac{d [ C ]}{d t} = - k ( \frac{[ E C ]}{1 + σ} ) \times ( \frac{{K}_{C\_Cb}}{[ Cb ] + {K}_{C\_Cb}} ) \times ( \frac{{K}_{C\_Op}}{[ Op ] + {K}_{C\_Op}} ) & ( 4 ) \\ \frac{d [ Cb ]}{d t} = {K}_{f 1} \frac{d [ C ]}{d t} - \frac{{K}_{Cb} [ Cb ] [ B ]}{{K}_{m} \times ( ( 1 + \frac{[ G ]}{{K}_{Cb\_G}} ) + [ Cb ] )} & ( 5 ) \\ \frac{d [ G ]}{d t} = ( {K}_{f 1} \frac{d [ C ]}{d t} - \frac{d [ Cb ]}{d t} ) {K}_{f 2} - \frac{1}{{Y}_{X\_G}} \frac{d [ X ]}{d t} & ( 6 ) \\ \frac{d [ X ]}{d t} = \frac{{μ}_{max} [ X ] [ G ]}{[ G ] + {K}_{G}} \times ( 1 - \frac{[ Op ]}{{K}_{X\_Op}} ) & ( 7 ) \\ \frac{d [ Op ]}{d t} = ( \frac{{Y}_{Op\_G}}{{Y}_{X\_G}} ) \times \frac{d [ X ]}{d t} & ( 8 )\end{matrix}$$

where:
   K_{C}_{_}_{Cb}=Inhibition constant of cellobiose on cellulose conversion [g/L]
   K_{C}_{_}_{Op}=Inhibition constant of organic product on cellulose conversion [g/L]
   K_{Cb}=Rate constant for hydrolysis of cellobiose to glucose [g/L]
   K_{Cb}_{_}_{G}=Inhibition of hydrolysis of cellobiose by glucose [g/L]
   K=Equilibrium constant of enzyme [L/g]
   k=Hydrolysis rate constant of enzyme [h⁻¹]
   k_{fc}=Enzyme adsorption constant to cellulose [h⁻¹]
   K_{G}=Monod constant [g/L]
   Kₘ=Michaelis constant of enzyme for cellobiose [g/L]
   K_{X}_{_}_{Op}=Inhibition of cell growth by organic product [g/L]
   Y_{Op}_{_}_{G}=Yield of organic product cells per gram of glucose
   Y_{X}_{_}_{G}=Yield of organism cells per gram of glucose
   μₘₐₓ=Maximum growth rate of organism cells [h⁻¹]
   σₑ=Maximum bonding capacity of enzyme [dimensionless]

The processor receives a further input from a sensor in the bioreactor of the total actual organic product and compares the total calculated organic product and the total actual organic product. The processor then transmitting an instruction to an agitator associated with the bioreactor to control agitation of the content of the bioreactor if the total actual organic product is outside a predetermined range of the total calculated organic product.

For example, organic product may be desired where the total actual organic product is within 10% of the total calculated organic product. If the processor calculates a total calculated organic product of 16 g/L, then the total actual organic product must be in the range 14.4 g/L to 17.6 g/L. If the total actual organic product produced in the bioreactor is outside this range, e.g. 13 g/L, the processor transmits an instruction to the agitator associated with the bioreactor to control agitation of the content of the bioreactor. Agitation of the content of the bioreactor may be achieved using any suitable method, for example through an impeller, propeller, turbine anchor, gas induction or the like.

The processor may also transmit instructions to control one or more of temperature, pH, and pressure in the bioreactor if the total actual organic product is outside the predetermined range of the total calculated organic product. For example, the processor may transmit an instruction to a heater associated with the bioreactor to control temperature in the bioreactor, the processor may transmit an instruction to an inlet valve of the bioreactor to control addition of an acid or base to control pH in the bioreactor, and the processor may transmit an instruction to a pressurizing component associated with the bioreactor to control pressure in the bioreactor.

The temperature, pH, and pressure may be controlled within predetermined ranges. For example, temperature in the bioreactor may be controlled within a predetermined temperature range e.g. between 32° C. and 45° C. If temperature in the bioreactor falls outside this temperature range, e.g. 50° C., the processor may transmit an instruction to a heater associated with the bioreactor so as to turn off heating. The processor may also transmit an instruction to a cooling coil for the bioreactor so as to cool the content of the bioreactor. If temperature in the bioreactor falls outside this temperature range, this may suggest overgrowth of the organism in the bioreactor. The processor may be configured to transmit a further instruction to an outlet valve of the bioreactor to cause purging of cellulose from the bioreactor. Purging of cellulose from the bioreactor will starve the organism and limit the growth of the organism.

The processor may receive inputs from a plurality of sensors in the bioreactor. The inputs may be, for example, measurements of enzyme loading concentration, cellulose concentration and organism concentration. The processor may then use the inputs from the plurality of sensors to calculate conversion of cellulose and provide the total calculated organic product.

The processor may also receive additional inputs such as measurements relating to the rate of formation of enzyme-substrate complexes and oxygen supplied to the bioreactor and may calculate conversion of cellulose to provide the total calculated organic product using these additional inputs. For example, equation (1) relating to the rate of formation of enzyme-substrate complexes may be substituted by the additional input received by the processor.

Controlling agitation of the content of the bioreactor and one or more of temperature, pH, or pressure will ensure that optimum efficiencies are achieved.

Modelling

The numerical model used to configure a processor of a computing device for simultaneous saccharification and fermentation of crystalline cellulose assumes the following pathway from substrate to product:

Enzymes [E] absorb to the cellulose [C] particle surface forming enzyme-substrate complexes [EC]. The rate of formation of these bonds is described by a dynamic adsorption type equation which correlates adsorbed enzyme with the conversion rate of the substrate

$$\frac{d [ E C ]}{d t} = \frac{d [ C ]}{d t} ( 1 + {σ}_{e} ) + {k}_{fc} [ {E}_{f} ] [ {C}_{f} ] ( 1 + {σ}_{e} ) - \frac{{k}_{fc}}{K} [ E C ] \qquad ( 1 )$$

with σₑ representing the maximum bonding capacity of the specific enzyme. This equation can be adjusted and terms adding depending on the enzyme used and the enzyme complex formed.

The concentration of free enzymes [E_{f}] and concentration of free cellulose [C_{f}] in the medium are determined from

$$\begin{matrix}[ {E}_{f} ] = [ {E}_{T} ] - \frac{[ E C ] \times σ}{( 1 + σ )} & ( 2 ) \\ [ {C}_{f} ] = [ {C}_{T} ] - \frac{[ E C ]}{( 1 + σ )} & ( 3 )\end{matrix}$$

where [E_{T}] and [C_{T}] represents the total available enzyme and cellulose at a given time and σ_{c} represents the maximum enzyme capacity of the substrate (g enzymes/g cellulose).

The conversion of cellulose is described using equation (4) with the formation of cellobiose [Cb] and subsequent glucose [G] described in equations (5) and (6).

$$\begin{matrix}\frac{d [ C ]}{d t} = - k ( \frac{[ E C ]}{1 + σ} ) \times ( \frac{{K}_{C\_Cb}}{[ Cb ] + {K}_{C\_Cb}} ) \times ( \frac{{K}_{C\_Op}}{[ Op ] + {K}_{C\_Op}} ) & ( 4 ) \\ \frac{d [ Cb ]}{d t} = {K}_{f 1} \frac{d [ C ]}{d t} - \frac{{K}_{Cb} [ Cb ] [ B ]}{{K}_{m} \times ( ( 1 + \frac{[ G ]}{{K}_{Cb\_G}} ) + [ Cb ] )} & ( 5 ) \\ \frac{d [ G ]}{d t} = ( {K}_{f 1} \frac{d [ C ]}{d t} - \frac{d [ Cb ]}{d t} ) {K}_{f 2} - \frac{1}{{Y}_{X\_G}} \frac{d [ X ]}{d t} & ( 6 )\end{matrix}$$

Growth of the organism is described using equation (7) with subsequent production rate for an organic product described using equation (8).

$$\begin{matrix}\frac{d [ X ]}{d t} = \frac{{μ}_{max} [ X ] [ G ]}{[ G ] + {K}_{G}} \times ( 1 - \frac{[ Op ]}{{K}_{X\_Op}} ) & ( 7 ) \\ \frac{d [ Op ]}{d t} = ( \frac{{Y}_{Op\_G}}{{Y}_{X\_G}} ) \times \frac{d [ X ]}{d t} & ( 8 )\end{matrix}$$

Controlling the conversion of crystalline insoluble cellulose to an organic product may also be achieved by monitoring suspension of particles in the medium in which the conversion of crystalline insoluble cellulose takes place. Solute particles in the medium may not dissolve but get suspended throughout the bulk of the medium. The particles may be suspended throughout the medium by any suitable agitator, for example, an impeller, a propeller, a turbine, an anchor, gas induction, or the like. The suspended particles will eventually settle if left undisturbed, therefore reducing efficiencies in the bioreactor.

The processor of the computing device may receive an input relating to the degree of settling of particles in the medium in which the conversion of crystalline insoluble cellulose takes place. The input may be luminous intensity of light backscattered by particles when light is sent through the medium. The backscattering intensity is directly proportional to the size and volume fraction of the particles. The input may also be a measurement of the number of particles in the bioreactor.

The processor may compare the input to a predetermined settling threshold. The settling threshold may be luminous intensity in a range, e.g. 0.2 cd to 0.8 cd, from the top of the medium to the bottom. If the backscattering intensity falls outside this range, e.g. 0.9 cd at the bottom of the medium, the processor transmits an instruction to the agitator associated with the bioreactor to control agitation of the content of the bioreactor thereby suspending the particles again throughout the medium.

Controlling agitation of the content of the bioreactor to ensure particles are sufficiently suspended in the medium will ensure that optimum efficiencies are achieved.

Specific embodiments of the invention are now described in greater detail with reference to the Figures.

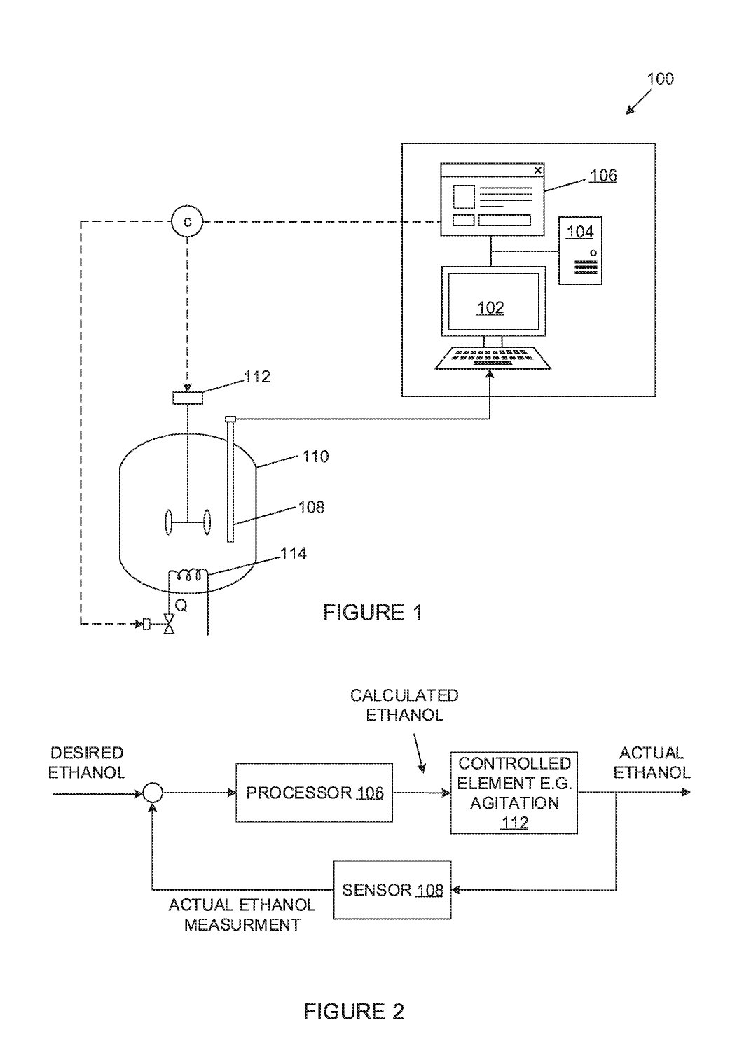

FIG. 1 is a schematic diagram of a system (100) for controlling the conversion of a crystalline insoluble cellulose, to an organic product according to embodiments of the present invention. The system (100) includes a computing device (102) which has a memory (104) for storing computer-readable program code and a processor (106) for executing the computer-readable program code. The processor (106) is configured to interact with one or more sensors (108) in a bioreactor (110) and an agitator (112) for the bioreactor (110). The agitator (108) may employ any suitable method for agitating the content of the bioreactor (110), for example, an impeller, a propeller, a turbine, an anchor, gas induction, or the like.

In this embodiment Avicel, a crystalline insoluble cellulose, is converted to ethanol and the culture medium in the bioreactor (110) includes Saccharomyces cerevisiae (also known as Bakers' yeast).

The processor (106) receives the following inputs from one or more sensors (108) in the bioreactor (110):
Yeast cell concentration [g/L]—([X])
Cellulose concentration [g/L]—([C])
Cellobiose concentration [g/L]—([Cb])
Exo-cellulase enzyme concentration [g/L]—([Eₑₓₒ])
Endo-cellulase enzyme concentration [g/L]—([E_{endo}])
β-Glucosidase concentration [g/L]—([B])
Cellulose-enzyme complex concentration [g/L]—([EC]ₑₓₒ),
Cellulose-enzyme complex concentration [g/L]—([EC]_{endo}),
Ethanol concentration [g/L]—([Eth])
Glucose concentration [g/L]—([G]),

The above inputs may alternatively be provided by an operator.

The processor (106) calculates conversion of cellulose using these inputs to provide a total calculated ethanol in the bioreactor (110). The processor (106) calculates conversion of cellulose by solving the following equations:

$$\begin{matrix}\frac{d {[ E C ]}_{endo}}{d t} = \frac{d {[ C ]}_{endo}}{d t} \times ( 1 + {σ}_{endo} ) + {k}_{fc} [ {E}_{f , endo} ] [ {C}_{f , endo} ] ( 1 + {σ}_{endo} ) - {\frac{{k}_{fc}}{{K}_{endo}} [ E C ]}_{endo} & ( 9 ) \\ \frac{d {[ E C ]}_{exo}}{d t} = \frac{d {[ C ]}_{exo}}{d t} \times ( 1 + {σ}_{exo} ) + {k}_{fc} [ {E}_{f , exo} ] [ {C}_{f , exo} ] ( 1 + {σ}_{exo} ) - {\frac{{k}_{fc}}{{K}_{exo}} [ E C ]}_{exo} & ( 10 ) \\ [ {E}_{f} ] = [ {E}_{T} ] - \frac{[ E C ] \times σ}{( 1 + σ )} & ( 11 ) \\ [ {C}_{f} ] = [ {C}_{T} ] - \frac{[ E C ]}{( 1 + σ )} & ( 12 ) \\ \frac{d {[ C ]}_{endo}}{d t} = - {k}_{endo} \times \frac{{[ E C ]}_{endo}}{1 + {σ}_{endo}} \times ( \frac{{K}_{C\_Cb}}{[ Cb ] + {K}_{C\_Cb}} ) \times ( \frac{{K}_{C\_Eth}}{[ Eth ] + {K}_{C\_Eth}} ) & ( 13 ) \\ \frac{d {[ C ]}_{exo}}{d t} = tanh ( \frac{t}{τ} ) \times - {k}_{exo} \times \frac{{[ E C ]}_{exo}}{1 + {σ}_{exo}} \times ( \frac{{K}_{C\_Cb}}{[ Cb ] + {K}_{C\_Cb}} ) \times ( \frac{{K}_{C\_Eth}}{[ Eth ] + {K}_{C\_Eth}} ) & ( 14 ) \\ \frac{d [ Cb ]}{d t} = - \frac{342}{324} \times \frac{d [ C ]}{d t} - \frac{{K}_{Cb} [ Cb ] [ B ]}{{K}_{m} \times ( ( 1 + \frac{[ G ]}{{K}_{Cb\_G}} ) + [ Cb ] )} & ( 15 ) \\ \frac{d [ G ]}{d t} = ( - \frac{342}{324} \times \frac{d [ C ]}{d t} - \frac{d [ Cb ]}{d t} ) \times \frac{360}{342} - \frac{1}{{Y}_{X\_G}} \times \frac{d [ X ]}{d t} & ( 16 ) \\ \frac{d [ X ]}{d t} = \frac{{μ}_{max} [ X ] [ G ]}{[ G ] + {K}_{G}} \times ( 1 - \frac{[ Eth ]}{{K}_{X\_Eth}} ) & ( 17 ) \\ \frac{d [ Eth ]}{d t} = ( \frac{{Y}_{Eth\_G}}{{Y}_{X\_G}} ) \times \frac{d [ X ]}{d t} & ( 18 )\end{matrix}$$

where:
   K_{C}_{_}_{Cb}=Inhibition constant of cellobiose on cellulose conversion [g/L]
   K_{C}_{_}_{Eth}=Inhibition constant of ethanol on cellulose conversion [g/L]
   K_{Cb}=Rate constant for hydrolysis of cellobiose to glucose [g/L]
   K_{Cb}_{_}_{G}=Inhibition of hydrolysis of cellobiose by glucose [g/L]
   K_{endo}=Equilibrium constant for endoglucanase [L/g]
   k_{endo}=Hydrolysis rate constant of endoglucanase [h⁻¹]
   Kₑₓₒ=Equilibrium constant for exoglucanase [L/g]
   kₑₓₒ=Hydrolysis rate constant of exoglucanase [h⁻¹]
   k_{fc}=Enzyme adsorption constant to Avicel [h⁻¹]
   K_{G}=Monod constant [g/L]
   Kₘ=Michaelis constant of β-glucosidase for cellobiose [g/L]
   K_{X}_{_}_{Eth}=Inhibition of cell growth by ethanol [g/L]
   Y_{Eth}_{_}_{G}=Yield of ethanol cells per gram of glucose
   Y_{X}_{_}_{G}=Yield of yeast cells per gram of glucose
   μₘₐₓ=Maximum growth rate of yeast cells [h⁻¹]
   σ_{endo}=Endoglucanse enzyme capacity on Avicel [dimensionless]
   σₑₓₒ=Exoglucanase enzyme capacity on Avicel [dimensionless]
   τ=Time Constant [h]

The processor (106) receives a further input from a sensor (108) in the bioreactor (110) of the total actual ethanol in the bioreactor (110) and compares the total calculated ethanol and the total actual ethanol. The processor (106) then transmits an instruction to an agitator (112) associated with the bioreactor (110) to control agitation of the content of the bioreactor (110) if the total actual ethanol is outside a predetermined range of the total calculated ethanol.

For example, FIG. 2 is a block diagram which illustrates controlling agitation of the content of the bioreactor according to the present embodiment. Ethanol may be desired where the total actual ethanol is within 10% of the total calculated ethanol. If the processor (106) calculates a total calculated ethanol of 16 g/L, then the total actual ethanol must be in the range 14.4 g/L to 17.6 g/L. If the total actual ethanol produced in the bioreactor (110) is outside this range, e.g. 13 g/L, the processor (106) transmits an instruction to an agitator (112) associated with the bioreactor (110) to control agitation of the content of the bioreactor (110).

The processor (106) may transmit instructions to control one or more of temperature, pH, and pressure in the bioreactor if the total actual ethanol is outside the predetermined range of the total calculated ethanol. For example, the processor (106) may transmit an instruction to a heater (114) associated with the bioreactor (110) to control temperature in the bioreactor (110), or may transmit an instruction to an inlet valve of the bioreactor (110) to control addition of an acid or base so as to control pH in the bioreactor (110), or may transmit an instruction to a pressurizing component associated with the bioreactor (110) to control pressure in the bioreactor (110).

The temperature, pH, and pressure may be controlled within predetermined ranges. Temperature in the bioreactor (110) may, for example, be controlled within a predetermined range of between 32° C. and 45° C. If temperature in the bioreactor (110) falls outside this range, e.g. 50° C., the processor (106) may transmit an instruction to the heater (114) associated with the bioreactor (110) so as to turn off heating. The processor (106) may transmit an instruction to a cooling coil for the bioreactor (110) so as to cool the content of the bioreactor (110).

Controlling agitation of the content of the bioreactor (110), temperature, pH, and pressure will ensure that optimum efficiencies are achieved in the bioreactor (110).

Modelling

The numerical model used to configure the processor (106) for simultaneous saccharification and fermentation of crystalline cellulose to ethanol assumes the following pathway from substrate to product:

Endoglucanase and exoglucanase enzymes adsorb to the insoluble Avicel particle surface forming enzyme-substrate complexes [EC]_{endo} and [EC]ₑₓₒ. The rate of formation of these bonds is described by dynamic adsorption type equations which correlates adsorbed enzyme with the conversion rate of the substrate.

$$\begin{matrix}\frac{d {[ E C ]}_{endo}}{d t} = \frac{d {[ C ]}_{endo}}{d t} \times ( 1 + {σ}_{endo} ) + {k}_{fc} [ {E}_{f , endo} ] [ {C}_{f , endo} ] ( 1 + {σ}_{endo} ) - {\frac{{k}_{fc}}{{K}_{endo}} [ E C ]}_{endo} & ( 9 ) \\ \frac{d {[ E C ]}_{exo}}{d t} = \frac{d {[ C ]}_{exo}}{d t} \times ( 1 + {σ}_{exo} ) + {k}_{fc} [ {E}_{f , exo} ] [ {C}_{f , exo} ] ( 1 + {σ}_{exo} ) - {\frac{{k}_{fc}}{{K}_{exo}} [ E C ]}_{exo} & ( 10 )\end{matrix}$$

Where K_{endo} and Kₑₓₒ are adsorption affinity constants and the free enzymes [E_{f}] and free cellulose [C_{f}] are determined from

$$\begin{matrix}[ {E}_{f} ] = [ {E}_{T} ] - \frac{[ E C ] \times σ}{( 1 + σ )} & ( 11 ) \\ [ {C}_{f} ] = [ {C}_{T} ] - \frac{[ E C ]}{( 1 + σ )} & ( 12 )\end{matrix}$$

with σ being the maximum enzyme capacity of the substrate (g enzymes/g cellulose).

Hydrolysis of cellulose consisting of amorphous and crystalline structures is determined as a function of adsorbed enzyme [E_{C}] to the substrate and the enzymes specific enzyme activity (k_{endo} or kₑₓₒ):

$$\begin{matrix}\frac{d {[ C ]}_{endo}}{d t} = - {k}_{endo} \times \frac{{[ E C ]}_{endo}}{1 + {σ}_{endo}} \times ( \frac{{K}_{C\_Cb}}{[ Cb ] + {K}_{C\_Cb}} ) \times ( \frac{{K}_{C\_Eth}}{[ Eth ] + {K}_{C\_Eth}} ) & ( 13 ) \\ \frac{d {[ C ]}_{exo}}{d t} = tanh ( \frac{t}{τ} ) \times - {k}_{exo } \times \frac{{[ E C ]}_{exo}}{1 + {σ}_{exo}} \times ( \frac{{K}_{C\_Cb}}{[ Cb ] + {K}_{C\_Cb}} ) \times ( \frac{{K}_{C\_Eth}}{[ Eth ] + {K}_{C\_Eth}} ) & ( 14 )\end{matrix}$$

Inhibition from cellobiose and ethanol are calculated with correlations from Phillippidis et al. (1992).

It is assumed that cellulose chains are converted to cellobiose by exoglucanase. This conversion of cellulose to cellobiose is modelled proportionally to the cellulose hydrolysis rate, whereas the conversion of cellobiose to glucose is modelled using Michaelis-Menten kinetics as described by Phillippidis et al. (1992).

$$\frac{d [ Cb ]}{d t} = - \frac{342}{324} \times \frac{d [ C ]}{d t} - \frac{{K}_{Cb} [ Cb ] [ B ]}{{K}_{m} \times ( ( 1 + \frac{[ G ]}{{K}_{Cb\_G}} ) + [ Cb ] )} \qquad ( 15 )$$

Hydrolysis of cellobiose to glucose by β-glucosidase and the glucose utilization by the yeast cells can be described by equation:

$$\frac{d [ G ]}{d t} = ( - \frac{342}{324} \times \frac{d [ C ]}{d t} - \frac{d [ Cb ]}{d t} ) \times \frac{360}{342} - \frac{1}{{Y}_{X\_G}} \times \frac{d [ X ]}{d t} \qquad ( 16 )$$

The fermentation of glucose to ethanol, is modelled as an anaerobic batch process following the stoichiometric approximation that describes the catabolic conversion of glucose

C₆H₁₂O₆+0.2H₂→1.8(C₂H₆O+CO₂)+0.2C₃H₈O₃

The yeast growth rate and product production rate for ethanol is thus described by:

$$\begin{matrix}\frac{d [ X ]}{d t} = \frac{{μ}_{max} [ X ] [ G ]}{[ G ] + {K}_{G}} \times ( 1 - \frac{[ Eth ]}{{K}_{X\_Eth}} ) & ( 17 ) \\ \frac{d [ Eth ]}{d t} = ( \frac{{Y}_{Eth\_G}}{{Y}_{X\_G}} ) \times \frac{d [ X ]}{d t} & ( 18 )\end{matrix}$$

In another embodiment, Avicel is converted to glycerol and the culture medium in the bioreactor (110) includes Saccharomyces cerevisiae or Bakers' yeast.

The processor (106) receives the following inputs from the sensors (108) in the bioreactor (110):
Yeast cell concentration [g/L]—([X])
Cellulose concentration [g/L]—([C])
Cellobiose concentration [g/L]—([Cb])
Exo-cellulase enzyme concentration [g/L]—([Eₑₓₒ])
Endo-cellulase enzyme concentration [g/L]—([E_{endo}])
β-Glucosidase concentration [g/L]—([B])
Cellulose-enzyme complex concentration [g/L]—([EC]ₑₓₒ),
Cellulose-enzyme complex concentration [g/L]—([EC]_{endo}),
Glycerol concentration [g/L]—([Gly])
Glucose concentration [g/L]—([G])

As mentioned above, the inputs may alternatively be provided by an operator.

The processor (106) calculates conversion of cellulose using these inputs to provide a total calculated glycerol in the bioreactor (110). The processor (106) calculates conversion of cellulose by solving the following equations:

$$\begin{matrix}\frac{d {[ E C ]}_{endo}}{d t} = \frac{d {[ C ]}_{endo}}{d t} \times ( 1 + {σ}_{endo} ) + {k}_{fc} [ {E}_{f , endo} ] [ {C}_{f , endo} ] ( 1 + {σ}_{endo} ) - {\frac{{k}_{fc}}{{K}_{endo}} [ E C ]}_{endo} & ( 9 ) \\ \frac{d {[ E C ]}_{exo}}{d t} = \frac{d {[ C ]}_{exo}}{d t} \times ( 1 + {σ}_{exo} ) + {k}_{fc} [ {E}_{f , exo} ] [ {C}_{f , exo} ] ( 1 + {σ}_{exo} ) - {\frac{{k}_{fc}}{{K}_{exo}} [ E C ]}_{exo} & ( 10 ) \\ [ {E}_{f} ] = [ {E}_{T} ] - \frac{[ E C ] \times σ}{( 1 + σ )} & ( 11 ) \\ [ {C}_{f} ] = [ {C}_{T} ] - \frac{[ E C ]}{( 1 + σ )} & ( 12 ) \\ \frac{d {[ C ]}_{endo}}{d t} = - {k}_{endo} \times \frac{{[ E C ]}_{endo}}{1 + {σ}_{endo}} \times ( \frac{{K}_{C\_Cb}}{[ Cb ] + {K}_{C\_Cb}} ) \times ( \frac{{K}_{C\_Gly}}{[ Gly ] + {K}_{C\_Gly}} ) & ( 19 ) \\ \frac{d {[ C ]}_{exo}}{d t} = tanh ( \frac{t}{τ} ) \times - {k}_{exo} \times \frac{{[ E C ]}_{exo}}{1 + {σ}_{exo}} \times ( \frac{{K}_{C\_Cb}}{[ Cb ] + {K}_{C\_Cb}} ) \times ( \frac{{K}_{C\_Gly}}{[ Gly ] + {K}_{C\_Gly}} ) & ( 20 ) \\ \frac{d [ Cb ]}{d t} = - \frac{342}{324} \times \frac{d [ C ]}{d t} - \frac{{K}_{Cb} [ Cb ] [ B ]}{{K}_{m} \times ( ( 1 + \frac{[ G ]}{{K}_{Cb\_G}} ) + [ Cb ] )} & ( 15 ) \\ \frac{d [ G ]}{d t} = ( - \frac{342}{324} \times \frac{d [ C ]}{d t} - \frac{d [ Cb ]}{d t} ) \times \frac{360}{342} - \frac{1}{{Y}_{X\_G}} \times \frac{d [ X ]}{d t} & ( 16 ) \\ \frac{d [ X ]}{d t} = \frac{{μ}_{max} [ X ] [ G ]}{[ G ] + {K}_{G}} \times ( 1 - \frac{[ Gly ]}{{K}_{X\_Gly}} ) \\ \begin{matrix} \frac{d [ Gly ]}{d t} = ( \frac{{Y}_{Gly\_G}}{{Y}_{X\_G}} ) \times \frac{d [ X ]}{d t} & ( 22 )\end{matrix} & ( 21 )\end{matrix}$$

where:
   K_{C}_{_}_{Gly}=Inhibition constant of glycerol on cellulose conversion [g/L]
   K_{X}_{_}_{Gly}=Inhibition of cell growth by glycerol [g/L]
   Y_{Gly}_{_}_{G}=Yield of glycerol cells per gram of glucose

The processor (106) receives a further input from a sensor (108) in the bioreactor (110) of the total actual glycerol in the bioreactor (110) and compares the total calculated glycerol and the total actual glycerol. The processor (106) then transmits an instruction to an agitator (112) associated with the bioreactor (110) to control agitation of the content of the bioreactor (110) if the total actual glycerol is outside a predetermined range of the total calculated glycerol.

For example, glycerol may be desired where the total actual glycerol is within 10% of the total calculated glycerol. If the processor (106) calculates a total calculated glycerol of 16 g/L, then the total actual glycerol must be in the range 14.4 g/L to 17.6 g/L. If the total actual glycerol produced in the bioreactor (110) is outside this range, e.g. 13 g/L, the processor (106) transmits an instruction to an agitator (112) associated with the bioreactor (110) to control agitation of the content of the bioreactor (110).

The processor (106) may also initiate a signal that controls one or more of temperature, pH, and pressure in the bioreactor (110) when the total actual glycerol is outside the predetermined range of the total calculated glycerol. For example, the processor (106) may transmit an instruction to a heater (114) associated with the bioreactor (110) to control temperature in the bioreactor (110), or may transmit an instruction to an inlet valve of the bioreactor (110) to control addition of an acid or base so as to control pH in the bioreactor (110), or may transmit an instruction to a pressurizing component associated with the bioreactor (110) to control pressure in the bioreactor (110).

The temperature, pH, and pressure may be controlled within predetermined ranges. Temperature in the bioreactor (110) may, for example, be controlled within a predetermined range of between 32° C. and 45° C. If temperature in the bioreactor (110) falls outside this range, e.g. 50° C., the processor (106) may transmit an instruction to the heater (114) associated with the bioreactor (110) so as to turn off heating. The processor (106) may transmit an instruction to a cooling coil for the bioreactor (110) so as to cool the content of the bioreactor (110).

Controlling agitation of the content of the bioreactor (110), temperature, pH, and pressure will ensure that optimum efficiencies are achieved in the bioreactor (110).

The numerical model used to configure the processor (106) for simultaneous saccharification and fermentation of crystalline cellulose to glycerol is similar, mutatis mutandis, to that of ethanol as described above. For example the fermentation of glucose to glycerol, is also modelled as an anaerobic batch process following the stoichiometric approximation that describes the catabolic conversion of glucose

C₆H₁₂O₆+0.2H₂→1.8(C₂H₆O+CO₂)+0.2C₃H₈O₃

The yeast growth rate and production rate for glycerol is thus described by

$$\begin{matrix}\frac{d [ X ]}{d t} = \frac{{μ}_{max} [ X ] [ G ]}{[ G ] + {K}_{G}} \times ( 1 - \frac{[ Gly ]}{{K}_{X\_Gly}} ) & ( 21 ) \\ \frac{d [ Gly ]}{d t} = ( \frac{{Y}_{Gly\_G}}{{Y}_{X\_G}} ) \times \frac{d [ X ]}{d t} & ( 22 )\end{matrix}$$

It is appreciated that the processor (106) may receive further inputs from an operator to calculate rheological properties of the medium in which the conversion of crystalline insoluble cellulose takes place. The rheological properties of the medium may include drag, shear rates, wall shear stress, and flow fields required from computational fluid dynamics. The processor (106) may receive the following inputs which may be provided by an operator:
Inputs:
   Drag Coefficient [Dimensionless]—(CD)
   Lift coefficient [Dimensionless]—(CO
   Effective diameter of the particles [m]—(D_{eff})
   Gravitational constant [m/s²]—(g)
   Viscosity variable as a function of volume fraction [kg/m·s⁽¹⁻ⁿ⁾]—(K)
   Mass of the ethanol component [kg]—(mₑ)
   Mass of the glycerol component [kg]—(m_{g})
   Total mass of the solution [kg]—(mₜₒₜₐₗ)
   Mass of the water component [kg]—(m_{w})
   Viscosity power variable as a function of volume fraction—(n)
   Absolute temperature [K]—(T)
   Molar fraction of ethanol—(xₑ)
   Molar fraction of glycerol—(x_{g})
   Molar fraction of water—(x_{w})
   Volume fraction of the continuous phase—(α_{c})
   Volume fraction of the cellulose particles—(αₛ)
   Dynamic viscosity of mixture [kg/m·s]—(μ_{eff})
   Base dynamic viscosity of the fluid [kg/m·s]—(μₒ)
   Dynamic viscosity of base medium [kg/m·s]—(μ_{b})
   Continuous medium density [kg/m³]—(ρ_{eff})
   Particle density [kg/m³]—(ρₛ)

The processor (106) may calculate the rheological properties of the medium using these inputs by solving the following equations:

$$\begin{matrix} \frac{\partial }{\partial t} ( {α}_{i} {ρ}_{i} ) + ∇ \cdot ( {α}_{i} {ρ}_{i} {v}_{i} ) = 0 & ( 23 ) \\ \frac{\partial }{\partial t} ( {α}_{i} {ρ}_{i} {v}_{i} ) + ∇ \cdot ( {α}_{i} {ρ}_{i} {v}_{i} {v}_{i} ) = - {α}_{i} ∇ p + {α}_{i} {ρ}_{i} g + ∇ \cdot [ {α}_{i} ( {t}_{i} + {τ}_{i}^{t} ) ] + {M}_{i} & ( 24 ) \\ {F}_{L} = {C}_{L} {α}_{s} {ρ}_{c} [ {v}_{r} \times ( ∇ \times {v}_{r} ) ] & ( 25 ) \\ {F}_{cd}^{TD} = ( - {A}_{cs}^{D} ) \frac{{v}_{c}^{t}}{{σ}_{α}} ( \frac{∇ {α}_{s}}{{α}_{s}} - \frac{∇ {α}_{c}}{{α}_{c}} ) & ( 26 ) \\ {F}_{i , s} = - 101325 \{ tanh ⌊ 200 ( {α}_{max , s} - {α}_{s} ) ⌋ - 1 \} ∇ {α}_{s} & ( 27 ) \\ {F}_{cs}^{D} = {A}_{cs}^{D} ( {v}_{s} - {v}_{c} ) & ( 28 )\end{matrix}$$

with:

$$\begin{matrix} {A}_{cs}^{D} = \frac{3 {α}_{c} {α}_{s} {ρ}_{c} {C}_{D}}{4 {V}_{rs}^{2} {D}_{eff}} \vert {v}_{r} \vert & ( 29 ) \\ {V}_{rs} = 0.5 [ A - 0.06 {Re}_{s} + \sqrt{{( 0.06 {Re}_{s} )}^{2} + 0.12 {Re}_{s} ( 2 B - A ) + {A}^{2}} ] & ( 30 ) \\ {Re}_{s} = \frac{{ρ}_{c} {v}_{r} {D}_{eff}}{{μ}_{c}} & ( 31 ) \\ A = {α}_{c}^{4.14} & ( 32 ) \\ B = \{ \begin{matrix}0.8 {α}_{c}^{1.28} ; & {α}_{c} < {α}_{tr} \\ {α}_{c}^{2.65} ; & {α}_{c} \geq {α}_{tr}\end{matrix} & ( 33 ) \\ {C}_{D} = \frac{24}{{Re}_{s}} + \frac{6}{1 + \sqrt{{Re}_{s}}} + 0.4 \\ and & ( 34 ) \\ {μ}_{eff} = ( 1 - {α}_{s} ) {μ}_{0} + ( {α}_{s} ) {μ}_{s} \\ with \text{:} & ( 35 ) \\ {μ}_{0} = \{ {v}_{e / w} + a [ exp ( {bx}_{g} ) - 1 ] \} {ρ}_{eff} \\ with & ( 36 ) \\ {v}_{e / w} = {x}_{e} {v}_{e} + ( 1 - {x}_{e} ) {v}_{w} + {x}_{e} ( 1 - {x}_{e} ) {F}_{T} & ( 37 ) \\ {F}_{T} = [ exp ( \frac{3255}{T} - 9.41 ) + ( 1 - 2 {x}_{e} ) exp ( \frac{3917}{T} - 11.44 ) + {( 1 - 2 {x}_{e} )}^{2} exp ( \frac{5113}{T} - 16.6 ) ] & ( 38 ) \\ a = - 1.39 + 5.65 exp ( \frac{273.1 - T}{62.03} ) + [ 3.56 - \frac{89.18}{{( T - 273.1 )}^{1.5}} ] {x}_{e} - 8.80 {x}_{e}^{2} + 5.91 {x}_{e}^{3} & ( 39 ) \\ b = 4.11 + 5.54 exp ( \frac{273.1 - T}{25.03} ) & ( 40 ) \\ {ρ}_{eff} = \frac{{m}_{w} \times {ρ}_{w} + {m}_{e} \times {ρ}_{e} + {m}_{g} \times {ρ}_{G}}{{m}_{total}} & ( 41 ) \\ {μ}_{s} = K {\dot{γ}}^{n - 1} & ( 42 ) \\ K = \{ \begin{matrix}\frac{201 ( {α}_{s} - 0.0125 )}{[ 1 + 49 ( {α}_{s} - 0.0125 ) ]} ; & for {α}_{s} > 0.0125 \\ 0 ; & for {α}_{s} \leq 0.0125\end{matrix} & ( 43 ) \\ n = - 2.764 {α}_{s} - 0.631 & ( 44 )\end{matrix}$$

where:
F_{cs}^{D}=Drag Force [N/m³]
Mᵢ=Source terms [N/m³]
p=Pressure [Pa]
Reₛ=Reynolds number
V_{P, term}=Terminal settling velocity of the particles [m/s]
v_{c}=Velocity vector of the continuous phase [m/s]
vᵢ=Velocity vector of species [m/s]
vᵣ=Relative velocity vector [m/s]
vₛ=Velocity vector of the solids [m/s]
α_{c}=Volume fraction of the continuous phase
αᵢ=Volume fraction of the species
αₛ=Volume fraction of the cellulose particles
αₜᵣ=Volume fraction at which drag model transition occurs
μ=Dynamic viscosity of mixture [kg/m·s]
μₒ=Base dynamic viscosity of the fluid [kg/m·s]
μ_{c}=Dynamic viscosity adjustment for solids concentration [kg/m·s]
ρ_{c}=Density of continuous phase [kg/m³]
ρₑ=Density of ethanol [kg/m³]
ρ_{g}=Density of glycerol [kg/m³]
ρᵢ=Density of each species [kg/m³]
ρₚ=Particle density [kg/m³]
ρ_{w}=Density of water [kg/m³]
vₑ=Kinematic viscosity of ethanol [m²/s]
v=Kinematic viscosity of the aqueous ethanol-glycerol [m²/s]
v_{c/w}=Kinematic viscosity of the binary aqueous ethanol [m²/s]
v_{w}=Kinematic viscosity of water [m²/s]
τᵢ=Shear stress of species [N/m²]
\dot{γ}=Shear-rate [s⁻¹]
τᵢ¹=Turbulent shear stress of species [N/m²]
v_{c}^{t}=Turbulent kinematic viscosity of continuous phase [m²/s]
σ_{α}=Turbulent Prandtl number

The processor (106) may be configured to determine if the medium in which the conversion of crystalline insoluble cellulose takes place has Newtonian or non-Newtonian fluid behaviour using the calculated rheological properties. For example, the processor (106) may calculate the average viscosity of the medium and compare with a reference viscosity of RO water control of 8.31×10⁻⁴ kg/m·s. An average viscosity of 8.64×10⁻⁴±1% kg/m·s indicates a 3.8% increase in viscosity and Newtonian fluid behaviour of the medium in the bioreactor.

As organic product is produced, the organism involved in reactions in the bioreactor (110) grows and the viscosity of the medium increases. As the viscosity increases so too does the stress and strains applied to the agitator (112) associated with the bioreactor (110). Rapid agitation of the content of the bioreactor (110) may destroy the organism in the bioreactor (110). If the viscosity of the medium increases too quickly, immense force is needed to agitate the content of the bioreactor (110). The processor (106) may be configured to transmit instructions relating to the rate at which the content of the bioreactor (110) is agitated after determining whether the medium has Newtonian or non-Newtonian fluid behaviour. Further, as the organism grow, mechanical strain may be applied to the agitator (112) for the bioreactor (110). In this case, the processor (106) may transmit an instruction to an outlet valve of the bioreactor to cause purging of cellulose from the bioreactor so as to starve the organism and limit the growth of the organism.

Controlling agitation of the content of the bioreactor in such a case will ensure optimum energy usage and minimal damage to the agitator (112) or to the organism in the bioreactor (110).

Modelling

The flow-field and particle properties and fermentation medium conditions can be calculated using the following set of equations which would be solved iteratively by the processor in a three-dimensional domain:

Continuity is maintained throughout the domain by ensuring the conservation of mass:

$$\frac{\partial }{\partial t} ( {α}_{i} {ρ}_{i} ) + ∇ \cdot ( {α}_{i} {ρ}_{i} {v}_{i} ) = 0$$

Navier-Stokes equation calculated the momentum of the different species in the domain:

$$\frac{\partial }{\partial t} ( {α}_{i} {ρ}_{i} {v}_{i} ) + ∇ \cdot ( {α}_{i} {ρ}_{i} {v}_{i} {v}_{i} ) = - {α}_{i} ∇ p + {α}_{i} {ρ}_{i} g + ∇ \cdot [ {α}_{i} ( {τ}_{i} + {τ}_{i}^{t} ) ] + {M}_{i}$$

Lift force (Auton, 1988) which acts upon the cellulose particles:

F_{L}=C_{L}αₛρ_{c}[vᵣ×[vᵣ×(∇×vᵣ)]

Turbulent dispersion force which accounts for the effects of turbulence on the particle transportation.

$${F}_{cd}^{TD} = ( - {A}_{cs}^{D} ) \frac{{v}_{c}^{t}}{{σ}_{α}} ( \frac{∇ {α}_{s}}{{α}_{s}} - \frac{∇ {α}_{c}}{{α}_{c}} )$$

Solid pressure force limits the packing volume of the cellulose particles:

F_{i,s}=−101325{tan h[200(α_{max,s}−αₛ)]−1}∇αₛ

Particle drag force (Syamlal and O'brein, 1988) accounts for the interactions between the continuous phase and solid particles phases:

F_{cs}^{D}=A_{cs}^{D}(vₛ−v_{c})

with:

$$\begin{matrix}{A}_{cs}^{D} = \frac{3 {α}_{c} {α}_{s} {ρ}_{c} {C}_{D}}{4 {V}_{rs}^{2} {D}_{eff}} \vert {v}_{r} \vert \\ {V}_{rs} = 0.5 [ A - 0.06 {Re}_{s} + \sqrt{{( 0.06 {Re}_{s} )}^{2} + 0.12 {Re}_{s} ( 2 B - A ) + {A}^{2}} ] \\ {Re}_{s} = \frac{{ρ}_{c} {v}_{r} {D}_{eff}}{{μ}_{c}} \\ A = {α}_{c}^{4.14} \\ B = \{ \begin{matrix}0.8 {α}_{c}^{1.28} ; & {α}_{c} < {α}_{tr} \\ {α}_{c}^{2.65} ; & {α}_{c} \geq {α}_{tr}\end{matrix}\end{matrix}$$

Drag Coefficient (White, 1991):

$${C}_{D} = \frac{24}{{Re}_{s}} + \frac{6}{1 + \sqrt{{Re}_{s}}} + 0.4$$

Viscosity of the fermentation medium based on the particle ethanol and glycerol concentrations:

μ_{eff}=(1−αₛ)μ₀+(αₛ)μₛ

With (Moreira, 2009):

μ₀={v_{e/w}+a[exp(bx_{g})−1]}ρ_{eff}

with

$$\begin{matrix} {v}_{e / w} = {x}_{e} {v}_{e} + ( 1 - {x}_{e} ) {v}_{w} + {x}_{e} ( 1 - {x}_{e} ) {F}_{T} & \\ {F}_{T} = [ exp ( \frac{3255}{T} - 9.41 ) + ( 1 - 2 {x}_{e} ) exp ( \frac{3917}{T} - 11.44 ) + {( 1 - 2 {x}_{e} )}^{2} exp ( \frac{5113}{T} - 16.6 ) ] & \\ a = - 1.39 + 5.64 exp ( \frac{273.1 - T}{62.03} ) + [ 3.56 - \frac{89.18}{{( T - 273.1 )}^{1.5}} ] {x}_{e} - 8.80 {x}_{e}^{2} + 5.91 {x}_{e}^{3} & \\ b = 4.11 + 5.54 exp ( \frac{273.1 - T}{25.03} ) & \\ {ρ}_{eff} = \frac{{m}_{w} \times {ρ}_{w} + {m}_{e} \times {ρ}_{e} + {m}_{g} \times {ρ}_{G}}{{m}_{total}} & \\ {μ}_{S} = K {\dot{γ}}^{n - 1} & \\ K = \{ \begin{matrix}\frac{201 ( {α}_{s} - 0.0125 )}{[ 1 + 49 ( {α}_{s} - 0.0125 ) ]} ; & for {α}_{s} > 0.0125 \\ 0 ; & for {α}_{s} \leq 0.0125\end{matrix} & \\ n = - 2.764 {α}_{s} - 0.631 & \end{matrix}$$

Testing

Glucose Fermentations

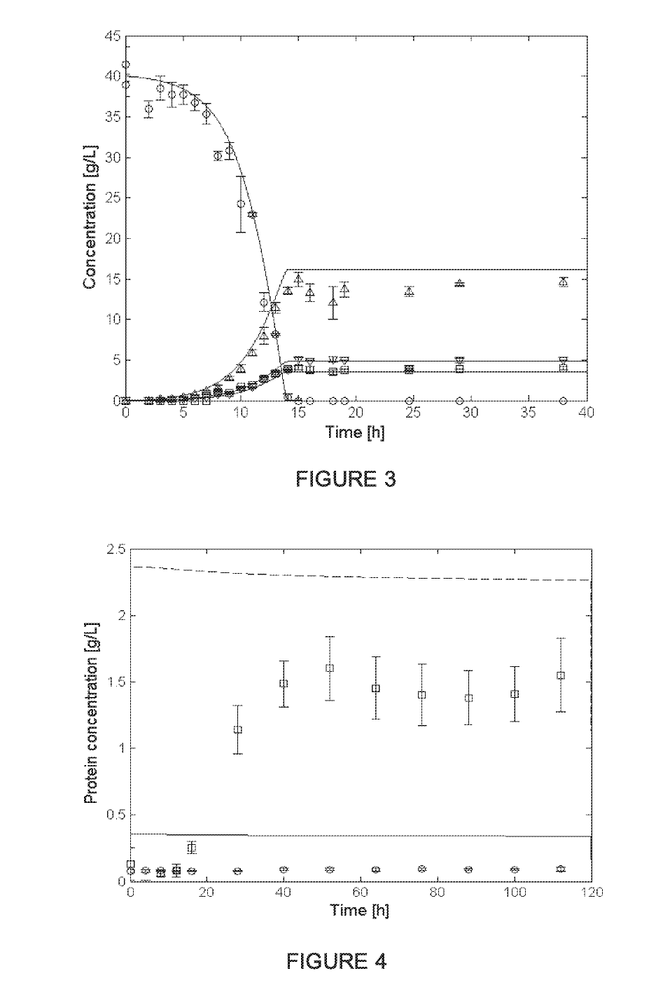

To verify the numerical model and system for Saccharomyces cerevisiae, anoxic fermentations were conducted at a glucose concentration of 40 g/L as shown in FIG. 3. The glucose fermentations utilizing S. cerevisiae provided specific growth rate of the strain MH1000 with glucose (◯), glycerol (□), ethanol (Δ) and yeast cells (∇). The calculated results are shown with solid lines.

The utilization and conversion of glucose by the yeast to form ethanol, glycerol and carbon dioxide was modelled as an anaerobic batch process following the stoichiometric approximation that describes the catabolic conversion of glucose

C₆H₁₂O₆+0.2H₂→1.8(C₂H₆O+CO₂)+0.2C₃H₈O₃

The maximum growth rate (μₘₐₓ) for this organism was calculated to be 0.38 h⁻¹.

Measured ethanol concentrations reached approximately 14.6 g/L (75% of the theoretical maximum). The numerical model, however, calculated a final ethanol concentration of 16.19 g/L.

A carbon balance was performed on the experimental results which indicated that 96.36%±0.24% of the carbon from the glucose was found in the fermentation products and biomass.

Discrepancies between the calculated and experimental results of ethanol concentration may indicate that a small portion of the ethanol evaporated from the reactor during the course of the experiment. This may also be deduced from the incomplete carbon balance of 96.36%.

Enzyme Activities

The enzyme activities and protein concentrations of Spezyme CP and Novozym 188 are summarised in Table 1 below:

TABLE 1

| Enzyme | FPU | CbU | Endoglucanase | Exoglucanase | β-glucosidase | Protein |
|---|---|---|---|---|---|---|
| Preparation | [U/mL] | [U/mL] | [IU/mL] | [IU/mL] | [IU/mL] | [mg/mL] |
| Spezyme^{CP} | 64.5 | N/A | 908.6 ± 90.5 | 1.447 ± 0.2 | 134.8 ± 3.9 | 195.4 ± 15.2 |
| Novozyme | N/A | 586.2 | 20.9 | N/A | 724.2 ± 35.8 | 148.1 ± 7.4 |
| 188 | | | | | | |

These values were used to estimate the added enzyme component in the medium in which the conversion of crystalline insoluble cellulose to ethanol occurs. According to Goyal (1991), 80% of the protein in a mixture derived from T. reesei such as Spezyme CP was identified as exoglucanase, whereas 12% was found to be endoglucanase. Filter paper units (FPU) and cellobiose units (CbU) were used to standardise and correlate the enzyme loadings with values provided from the literature.

The total enzyme protein added to each reactor for a cellulase loading of 10 FPU/g cellulose and a β-glucosidase loading 50 CbU/g cellulose amounts to a total initial concentration of 0.39 g/L endoglucanase, 2.59 g/L exoglucanase and 1.35 g/L of β-glucosidase.

The determined enzyme preparation activities of the Spezyme CP compared favourably with values found from literature with Kumar and Wyman (2008) reporting values of 59 FPU/mL and 123 mg/mL protein, with the mixture used in this study measured to be 64.5 FPU/mL with a protein concentration of 195.4 mg/mL. 10 FPU/mL was selected based on common practice from literature. 50 CbU/ml β-glucosidase was added to the solution to ensure that no cellobiose would accumulate in the reactors, which would severely inhibit the hydrolysis of the Avicel.

Enzymes Adsorption to Avicel

Avicel can be divided into two regions. One region is assumed to consist of long chains of cellulose with no exposed ends known as amorphous, which is randomly cut by the endoglucanase enzyme, creating new loose ends. Exoglucanase attaches to these ends and proceeds to hydrolyse the remaining densely packed crystalline chains into reduced sugars, primarily cellobiose. Both these regions are assumed to always be present in Avicel. An initial distribution of endoglucanase and exoglucanase binding sites was assumed as 55% and 45% respectively.

FIG. 4 shows adsorbed enzyme concentrations relative to initial loading free endoglucanase (∘) and exoglucanase (□) enzymes in solution. The total added endoglucanase (-) and exoglucanase (--) to the bioreactor are also shown. Adsorbed protein concentrations for endoglucanase and exoglucanase enzymes were calculated by subtracting the experimentally determined free enzyme concentrations in the broth from the theoretical total enzyme initially added (shown in FIG. 4). Experimental measurements further indicated that negligible amounts of β-glucosidase were adsorbed.

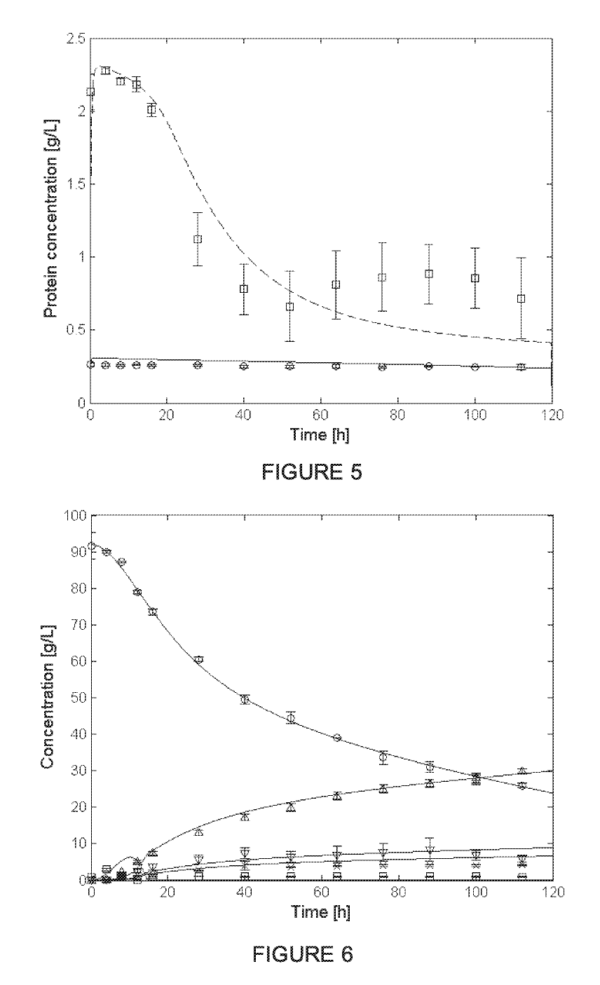

FIG. 5 shows adsorbed enzyme concentrations compared to numerical model adsorption of endoglucanase (∘) and exoglucanase (□) enzymes to Avicel. The calculated results for endoglucanase and exoglucanase adsorption are superimposed and indicated by solid lines. The calculated adsorbed enzymes concentrations indicate that adsorbed endoglucanase remained relatively consistent throughout the fermentation with adsorbed exoglucanase protein concentrations showing a considerable (5 fold) decrease from approximately 2.4 g/L to around 0.83 g/L after approximately 20 h (FIG. 4). Adsorption of endoglucanase and exoglucanase to Avicel was modelled using the dynamic adsorption models. With the assumed initial amorphous and crystalline constitution of Avicel, the models were capable of predicting the significant decrease in adsorbed exoglucanase. The model further correlates with the near constant adsorbed endoglucanase concentrations.

The adsorption models do not predict the apparent increase in adsorbed exoglucanase recorded after approximately 55 h. However, at a significance level of 5%, this apparent trend of increased adsorption is not statistically significant.

The adsorbed cellulases calculated from the difference in total and free cellulase in solution was compared with the results of the numerical model (FIG. 5). The adsorption model was capable of calculating the trends measured experimentally, but tends to under calculate the adsorbed exoglucanase concentrations during the later stages of the fermentation. The numerical model calculates the adsorbed endoglucanase concentrations reasonably well.

SSF of Avicel

FIG. 6 shows SSF of Avicel (◯) forming glucose (□) fermented to ethanol (Δ) and glycerol (x).

The growth of yeast cells (∇) is also shown and the calculated results are superimposed and indicated by solid lines. SSF of 100 g/L Avicel supplemented with Spezyme CP and Novozym 188 was conducted to verify the complete numerical model. Experimental results (FIG. 6) show that after 112 h, approximately 72.6% of the Avicel was converted. Furthermore, there appears to be a delay in the initial conversion of the Avicel (first 8 h) after which it is converted at a significantly higher rate. The numerical model does not predict this delay in enzymatic conversion and over predicts the glucose formed.

HPLC measurements indicated no trace of soluble cellobiose accumulation during the experiment, indicating that all cellulose was fully converted to glucose and fermented. The numerical model correctly predicts this rapid hydrolysis of cellobiose to glucose by β-glucosidase.

A small glucose peak of approximately 3 g/L was detected at approximately 4 h, thereafter rapidly decreasing to approximately 1 g/L for the remainder of the experiment. The numerical model calculates a glucose peak of 10.25 g/L at 7.4 h before the fermentation thereof the yeast decreases the concentration to 0 g/L.

Parameter fitting was performed on the remaining model constants for the SSF of Avicel. These values are presented in Table 2, with the specific hydrolyses rates k_{endo}, kₑₓₒ, equilibrium constant Kₑₓₒ, enzyme capacity σₑₓₒ and the yields Y_{CO2}_{_}_{G}, Y_{Eth}_{_}_{G} and Y_{Gly}_{_}_{G} determined empirically.

TABLE 2

| Symbol | Value | | Source |
|---|---|---|---|
| k_{endo} | 0.110 | h⁻¹ | This Work |
| kₑₓₒ | 0.07 | h⁻¹ | This Work |
| K_{endo} | 1.84 | L/g | Kumar and Wyman (2008) |
| Kₑₓₒ | 55 | L/g | This Work |
| k_{fc} | 1.8366 | L/(g · h) | Shao et al. (2008) |
| K_{C}_{_{—}}_{Cb} | 5.85 | g/L | Phillipidis et al. (1992) |
| K_{C}_{_{—}}_{Eth} | 50.35 | g/L | Phillipidis et al. (1992) |
| K_{Cb} | 0.02 | g/(U · h) | Gusakov and Sinitsyn (1985) |
| K_{Cb}_{_{—}}_{G} | 0.62 | g/L | Phillipidis et al. (1992) |
| K_{G} | 0.476 | g/L | Ghose and Tyagi (1979) |
| Kₘ | 10.56 | g/L | Phillipidis et al. (1992) |
| K_{X}_{_{—}}_{Eth} | 87 | g/L | Ghose and Tyagi (1979) |
| Y_{CO2}_{_{—}}_{G} | 0.4 | | This Work |
| Y_{Eth}_{_{—}}_{G} | 0.419 | | This Work |
| Y_{Gly}_{_{—}}_{G} | 0.091 | | This Work |
| Y_{X}_{_{—}}_{G} | 0.12 | | Ghose and Tyagi (1979) |
| μₘₐₓ | 0.4 | h⁻¹ | Ghose and Tyagi (1979) |
| σ_{endo} | 0.084 | | Kumar and Wyman (2008) |
| σₑₓₒ | 0.084 | | This Work |
| T | 8 | h | This Work |

The initial (<10 h) conversion rate of Avicel (FIG. 6) is found to be significantly lower than expected. The reason for this phenomenon is not clear and is not predicted by the numerical model. Possible explanations are that the enzymes are initially obstructed by other soluble constituents attached to the surface of the substrate which first needs to be cleared before the surface is significantly exposed for further adsorption. Once cleared, additional enzymes can attach and hydrolyze the cellulose causing the increase in conversion rate.

This initial delay in conversion rate measured experimentally explains the over prediction of the initial glucose peak (FIG. 6) calculated by the numerical model.

The specific cellulase activities for converting Avicel (k_{endo}, kₑₓₒ) along with the enzyme adsorption capacity (σₑₓₒ) and equilibrium constant (Kₑₓₒ) for exoglucanase were determined by parameter fitting from the numerical model.

Particle Properties

The particle density of the microcrystalline cellulose particles were determined using Archimedes principle as ρ_{P}=1605.7 kg/m³ with a standard deviation of 56.3 kg/m³. Particles settling experiments revealed an average terminal velocity of approximately V_{P, term}=6.53×10⁻³ m/s with a standard deviation of 3.44×10⁻³ m/s.

The average effective particle diameter (D_{eff}) was determined as D_{eff}=1.41×10⁻⁴ m with a standard deviation of 1.02×10⁻⁴ m. Using the known properties of water at 21° C., with μ=9.83×10⁻⁴ kg/m·s and ρ_{w}=998 kg/m³ ( [symbol] engel and Cimbala, 2006) along with D_{eff}=1.41×10⁻⁴ m, the Reynolds number was calculated as Re=0.9.

Viscosity

The viscosity of the base medium displayed Newtonian fluid behaviour with an average viscosity of 8.64×10⁻⁴±1% kg/m·s. The reference viscosity of the RO water control was 8.31×10⁴ kg/m·s, indicating a 3.8% increase. This increase is primarily attributed to the presence of the 17.5 g/L (NH₄)₂SO₄ in solution.

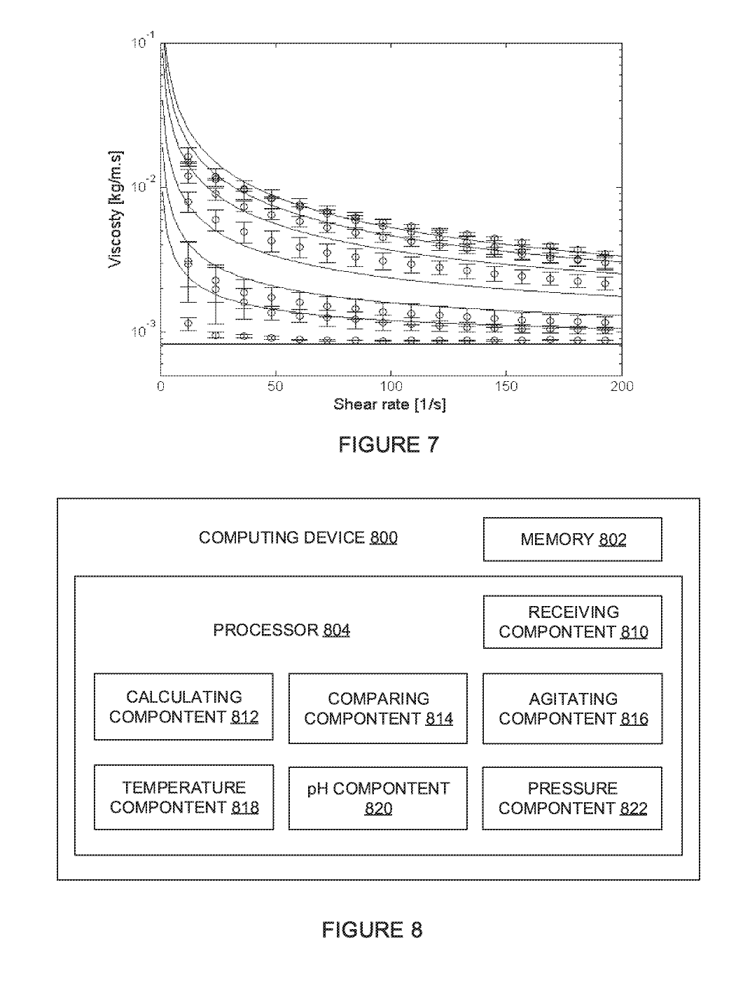

FIG. 7 shows dynamic viscosity for Avicel particles in water with the error-bars representing the standard deviation of each measurement (◯). The Avicel particles effect on the viscosity proved most significant as shown in FIG. 7. Particle concentrations of 100 g/L Avicel increased the viscosity to approximately 10⁻² kg/m·s decreasing with reduced concentrations as expected. The fluid viscosity with added particles displayed a shear-thinning effect in relation to the shear-rate (FIG. 7). Further investigation indicated that particle concentrations below 20 g/L had negligible effects on the viscosity of the medium and can be neglected.

The viscosity results from the oligosaccharides tests for both the Avicel particles in water and the hydrolysis experiments indicated no significant variation. The results from the Tween 80 test indicated no significant effect on the viscosity, except in the shear-rate range of 0 to 50 s⁻¹ where the average Tween 80 viscosity was 6%-26% lower than the control results.

Results for the ethanol and glycerol effects were calculated from equations 13 to 16 (ethanol) and found to increase the viscosity of the base medium to a maximum of 0.943×10⁻³ kg/m·s, with ethanol contributing most significantly.

The contribution of the yeast cells to the viscosity of the medium proved negligible as the total volume fraction occupied by the cells was calculated as 2.52×10⁻³, which equated to a relative viscosity increase of 0.6%.

Modelling

K and n variables were determined through the power-law regression methodology applied to the particle suspension viscosity measurements. The hyperbolic regression (Equation 43) best fitted the experimental values for K with a maximum error of 95% occurring at the concentration of 30 g/L (volume fraction of 0.0188) Avicel particles. The parameter fit for the n variable was linear (Equation 25) with a maximum error of 13%.

Applying the K and n numerical estimation parameters into equations 36 and 42, predictions for the effects of the particles on the dynamic viscosity (FIG. 7) displayed reasonable correlation with an average error of 11.1%. The largest error found in the final viscosity predictions was 26.4% found at the particle concentration of 30 g/L.

### CONCLUSION

Although there are inaccuracies with some calculations of the numerical method, the overall calculated rate of ethanol production correlates well with the experimental results. The numerical model enables enzymatic hydrolysis of cellulose and other sugar polymers to be modelled with a more direct methodology overcoming the limitation of curve fitting reaction rates to fit cellulose conversion models of the prior art.

It will be appreciated that the numerical model can be applied to many other enzymatic hydrolysis and fermentation processes of cellulose, hemicellulose and xylan in combination with fermenting bacteria, yeasts, and fungal organisms to form various organic products including but not limited to ethanol, glycerol, acetic acid, lactic acid, organic sugars, biomass, lignin and carboxylic acids such as pyruvate, lactate, malate, or succinate.

Also, the cellulase enzymes may be native or recombinant exoglucanases, endoglucanases and β-glucosidases from fungal or bacterial sources with minor adaptations to the numerical method. The mathematical formulas, or subsets of these, can be applied to enzymes active of hemicellulose (cellulose, xylan, mannan and galactans and derivates thereof), glucans, fructans, pectins and other carbohydrates available from plant biomass.

The microorganisms used for conversion of sugars to ethanol can be Saccharomyces cerevisiae MH1000 and can also be native or recombinant strains belonging to the genera Saccharomyces, Kluyveromyces, Candida, Pichia, Schizosaccharomyces, Hansenula, Kloeckera, Schwanniomyces, and Yarrowia. Particularly preferred yeast species as host cells include S. cerevisiae, S. bulderi, S. barnetti, S. exiguus, S. uvarum, S. diastaticus, S. carlsbergensis, K. lactis, K. marxianus, and K. fragilis.

The microorganisms used for conversion of sugars to a variety of organic products can be native or recombinant strains of a variety of bacteria, yeasts and fungi currently in use for the production of commercially important products from simple sugar streams.

A system as described in this disclosure with minor adaptations to the numerical method would allow real-time alterations of various process parameters, including temperature, pH, pressure, and the rate of agitation of the content of the bioreactor. This ensures that optimum efficiencies are achieved in the bioreactor during the production process for the organic products.

Minor additions to the numerical model may enable the processor to calculate additional results such as mechanical wear or over/underloading of the bioreactor. This can be achieved by the processor calculating the viscosity of the mixture in the bioreactor, for example using an input such as the concentration of cellulose and the following equations:

$$\begin{matrix}μ = ( 1 - {α}_{p} ) {μ}_{0} + ( {α}_{p} ) {μ}_{P} \\ Where , & \\ {μ}_{P} = K {\dot{γ}}^{n - 1} & \\ K = \{ \begin{matrix}\frac{201 ( {α}_{P} - 0.0125 )}{[ 1 + 49 ( {α}_{P} - 0.0125 ) ]} & for {α}_{P} > 0.0125 \\ 0 & for {α}_{P} \leq 0.0125\end{matrix} \} & \\ n = - 2.764 {α}_{P} + 0.369 & \end{matrix}$$

where μ₀ is viscosity of the base mixture, α_{P} is the particle concentration of cellulose and \dot{γ} is the shear rate.

For improved efficiencies and system robustness, additional sensors may be provided on an agitator for the bioreactor. These additional sensors may measure torque and rotation speed of the agitator. These additional sensors may provide further inputs to the processor which may be configured to alert operators should excessive stresses be detected on the system.

The system may further be used to determine the most efficient operative conditions for the bioreactor and may be combined with computational fluid dynamics which allow for prediction of more complicated biochemical processes, as not only reaction rates and product yields would be considered, but spatial effects as well, thus allowing more optimal design of bioreactor environments and thus minimising production costs.

Logical components of an exemplary computing device (800) used in the system are shown in FIG. 8. The computing device (800) may include at least one processor (804), a hardware module, or a circuit for executing the functions of the described components. The described components may be software units executing on the processor (804). Memory (802) may be configured to provide computer instructions to the processor (804) to carry out the functionality of the components. Some or all of the components may be provided by a software application installed onto and executable on the computing device (800). In some cases, for example in a cloud computing implementation, software units arranged to manage and/or process data on behalf of the computing device (800) may be provided remotely.

The processor (804) of the computing device (800) may include a receiving component (810), a calculating component (812), a comparing component (814) and an agitating component (816).

The receiving component (810) may be configured to receive an input from one or more sensors in the bioreactor. The input may be a measurement of one or more of concentration, temperature, pH and pressure. For example the receiving component (810) may receive the enzymatic loading concentration, substrate concentration and initial organism inoculation concentration as input. The receiving component (810) may also be configured to receive a further input from a sensor in the bioreactor of the total actual organic product. The receiving component (810) may further also be configured to receive additional inputs from sensors in the bioreactor such as measurements relating to the rate of formation of enzyme-substrate complexes and oxygen supplied to the bioreactor.

The calculating component (812) may be configured to calculate conversion of cellulose using an input to provide a total calculated organic product in the bioreactor. For example, in the exemplary embodiment in which Avicel is converted to ethanol, the calculating component (812) is configured to calculate the production rate of ethanol by iteratively solving equations (9) to (18). The calculating component (812) may also be configured to calculate rheological properties of the medium in which the conversion of crystalline insoluble cellulose to an organic product occurs, including calculating drag, shear rates and wall shear stress required, by for example solving equations (23) to (44).

The comparing component (814) may be configured to compare total calculated organic product and total actual organic product. For example, in the exemplary embodiment in which Avicel is converted to ethanol, the comparing component (814) may be configured to compare the total calculated ethanol and the total actual ethanol.

The agitating component (816) may be configured to transmit an instruction to an agitator associated with the bioreactor to control agitation of the content of the bioreactor. For example, in the exemplary embodiment in which Avicel is converted to ethanol, the agitation component (816) may be used to an instruction to the agitator associated with the bioreactor to control agitation of the content of the bioreactor.

The processor (804) may further include a temperature component (818), a pH component (820), and a pressure component (822).

The temperature component (818) may be configured to transmit an instruction to a heater associated with the bioreactor to control temperature in the bioreactor. For example, the temperature component (818) may be configured to control temperature in the bioreactor within a predetermined range throughout the production process e.g. between 32° C. and 45° C. The temperature component (818) may also be configured to transmit an instruction to an outlet valve of the bioreactor to cause purging of cellulose from the bioreactor if temperature is outside the predetermined temperature range. The temperature component (818) may further also be configured to transmit an instruction to the heater associated with the bioreactor to control temperature in the bioreactor if the total actual organic product is outside the predetermined range of the total calculated organic product.

The pH component (820) may be configured to transmit an instruction to an inlet valve of the bioreactor to control addition of an acid or base to control pH in the bioreactor. For example, the pH component (820) may be configured to maintain the pH in the bioreactor within a predetermined pH range throughout the production process e.g. between a pH of 5.4 and 5.6.

The pressure component (822) may be configured to transmit an instruction to a pressurizing component associated with the bioreactor to control pressure in the bioreactor. For example, the pressure component (820) may be configured to maintain the pressure in the bioreactor within a predetermined pressure range throughout the production process.

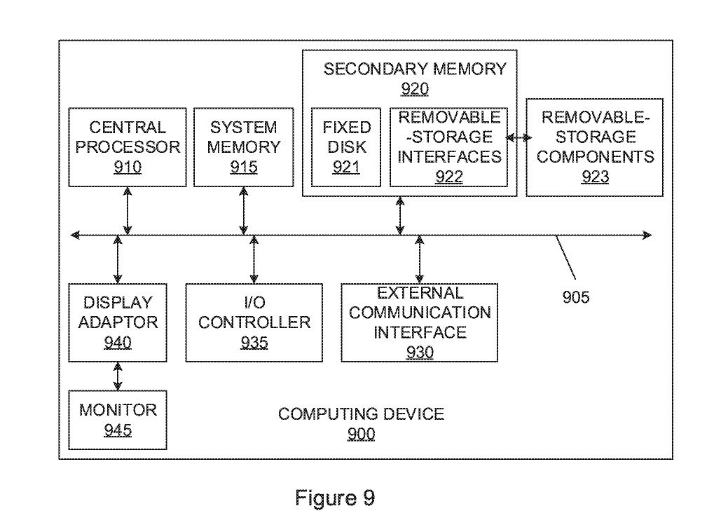

FIG. 9 is a block diagram which illustrates an example of a computing device (900) in which various aspects of the disclosure may be implemented. The computing device (900) may be suitable for storing and executing computer program code. The various participants and elements in the previously described system diagrams may use any suitable number of subsystems or components of the computing device (900) to facilitate the functions described herein.

The computing device (900) may include subsystems or components interconnected via a communication infrastructure (905) (for example, a communications bus, a cross-over bar device, or a network). The computing device (900) may include one or more central processors (910) and at least one memory component in the form of computer-readable media. In some configurations, a number of processors may be provided and may be arranged to carry out calculations simultaneously.

The memory components may include system memory (915), which may include read only memory (ROM) and random access memory (RAM). A basic input/output system (BIOS) may be stored in ROM. System software may be stored in the system memory (915) including operating system software.

The memory components may also include secondary memory (920). The secondary memory (920) may include a fixed disk (921), such as a hard disk drive, and, optionally, one or more removable-storage interfaces (922) for removable-storage components (923).

The removable-storage interfaces (922) may be in the form of removable-storage drives (for example, magnetic tape drives, optical disk drives, etc.) for corresponding removable storage-components (for example, a magnetic tape, an optical disk, etc.), which may be written to and read by the removable-storage drive.

The removable-storage interfaces (922) may also be in the form of ports or sockets for interfacing with other forms of removable-storage components (923) such as a flash memory drive, external hard drive, or removable memory chip, etc.

The computing device (900) may include an external communications interface (930) for operation of the computing device (900) in a networked environment enabling transfer of data between multiple computing devices (900). Data transferred via the external communications interface (930) may be in the form of signals, which may be electronic, electromagnetic, optical, radio, or other types of signal.

The external communications interface (930) may enable communication of data between the computing device (900) and other computing devices including servers and external storage facilities. Web services may be accessible by the computing device (900) via the communications interface (930).

The external communications interface (930) may also enable other forms of communication to and from the computing device (900) including, voice communication, near field communication, radio frequency communications, such as Bluetooth™, etc.

The computer-readable media in the form of the various memory components may provide storage of computer-executable instructions, data structures, program modules, software units, and other data. A computer program product may be provided by a computer-readable medium having stored computer-readable program code executable by the central processor (910). A computer program product may be provided by a non-transient computer-readable medium, or may be provided via a signal or other transient means via the communications interface (930).

Interconnection via the communication infrastructure (905) allows the central processor (910) to communicate with each subsystem or component and to control the execution of instructions from the memory components, as well as the exchange of information between subsystems or components.

Peripherals (such as printers, scanners, cameras, or the like) and input/output (I/O) devices (such as a mouse, touchpad, keyboard, microphone, and the like) may couple to the computing device (900) either directly or via an I/O controller (935). These components may be connected to the computing device (900) by any number of means known in the art, such as a serial port. One or more monitors (945) may be coupled via a display or video adapter (940) to the computing device (900).

The foregoing description of the embodiments of the invention has been presented for the purpose of illustration; it is not intended to be exhaustive or to limit the invention to the precise forms disclosed. Persons skilled in the relevant art can appreciate that many modifications and variations are possible in light of the above disclosure.

Some portions of this description describe the embodiments of the invention in terms of algorithms and symbolic representations of operations on information. These algorithmic descriptions and representations are commonly used by those skilled in the data processing arts to convey the substance of their work effectively to others skilled in the art. These operations, while described functionally, computationally, or logically, are understood to be implemented by computer programs or equivalent electrical circuits, microcode, or the like. The described operations may be embodied in software, firmware, hardware, or any combinations thereof.

It should be appreciated that components described herein may have the required configuration and/or arrangement of hardware, software, firmware, or the like for performing their associated functions, steps, processes, and/or operations. The software components or functions described in this application may be implemented as software code to be executed by one or more processors using any suitable computer language such as, for example, Java™, C++, or Perl™ using, for example, conventional or object-oriented techniques. The software code may be stored as a series of instructions, or commands on a non-transitory computer-readable medium, such as a random access memory (RAM), a read-only memory (ROM), a magnetic medium such as a hard-drive or an optical medium such as a CD-ROM. Any such computer-readable medium may also reside on or within a single computational apparatus, and may be present on or within different computational apparatuses within a system or network.

Any of the steps, operations, or processes described herein may be performed or implemented with one or more hardware or software modules, alone or in combination with other devices. In one embodiment, a software module is implemented with a computer program product comprising a non-transient computer-readable medium containing computer program code, which can be executed by a computer processor for performing any or all of the steps, operations, or processes described.

Finally, the language used in the specification has been principally selected for readability and instructional purposes, and it may not have been selected to delineate or circumscribe the inventive subject matter. It is therefore intended that the scope of the invention be limited not by this detailed description, but rather by any claims that issue on an application based hereon. Accordingly, the disclosure of the embodiments of the invention is intended to be illustrative, but not limiting, of the scope of the invention, which is set forth in the following claims.

Throughout the specification and claims unless the contents requires otherwise the word ‘comprise’ or variations such as ‘comprises’ or ‘comprising’ will be understood to imply the inclusion of a stated integer or group of integers but not the exclusion of any other integer or group of integers.

### REFERENCES

Albers E, Larsson C, Lidén G, Niklasson C, Gustafsson L. 2002. Continuous estimation of product concentration with calorimetry and gas analysis during anaerobic fermentations of Saccharomyces cerevisiae. Thermochimica Acta 394:185-190.
Auton T R, Hunt J C R, and Prud'homme M, 1988, The force exerted on a body in inviscid unsteady non-uniform rotational flow, Journal of Fluid Mechanics, 197:241-257.
Caminal G, Lopez-Satin J, Sola C. 1984. Kinetic modeling of the enzymatic hydrolysis of pretreated cellulose. Biotechnology and Bioengineering. 27: 1282-1290.
[symbol] engel Y. A. and Cimbala J. M. 2006. Fluid mechanics: fundamentals and applications. McGraw-Hill. New York.
Converse A O, Matsuno R, Tanaka M, Taniguchi M. 1988. A model of enzyme adsorption and hydrolysis of microcrystalline cellulose with slow deactivation of the adsorbed enzyme. Biotechnology and Bioengineering 32:38-45.
Converse A O, Optekar JD. 1993. A synergistic kinetics model for enzymatic cellulose hydrolysis compared to the degree-of-synergism experimental results. Biotechnology and Bioengineering 42:145-148.
Ghose T K, Tyagi R D. 1979. Rapid ethanol fermentation of cellulose hydrolysate. II. Product and substrate inhibition and optimization of fermentor design. Biotechnology and Bioengineering. 21: 1401-1420.
Goyal A, Ghosh B, Eveleigh D. 1991. Characteristics of fungal cellulases. Bioresource Technology 36:37-50.
Gusakov A V, Sinitsyn A P. 1985. Kinetics of the enzymatic hydrolysis of cellulose: 1. A mathematical model for a batch reactor process. Enzyme and Microbial Technology 7:346-352.
Kumar R, Wyman C E. 2008. An improved method to directly estimate cellulase adsorption on biomass solids. Enzyme and Microbial Technology 42:426-433.
Lee Y, Fan LT. 1982. Kinetic studies of enzymatic hydrolysis of insoluble cellulose: analysis of the initial rates. Biotechnology and Bioengineering. 24: 2383-2406.
Moreira, R, Chenlo, F and LeGall, D. 2009. Kinematic viscosity and refractive index of aqueous solutions of ethanol and glycerol. Industrial & Engineering Chemistry Research. 48:2157-2161.
Phillippidis G P, Smith T K, Wyman C E. 1992. Study of the enzymatic hydrolysis of cellulose for production of fuel ethanol by the simultaneous saccharification and fermentation process. Biotechnology and Bioengineering 41:846-853.
Scheiding W, Thoma M, Ross A, Schügerl K. 1984. Modelling of the enzymatic hydrolysis of cellobiose and cellulose by a complex enzyme mixture of Trichoderma reesei QM 9414. Applied Microbiology and Biotechnology 20:176-182.
Shao X, Lynd L R, Wyman C E, Bakker A. 2008. Kinetic modeling of cellulosic biomass to ethanol via simultaneous saccharification and fermentation: Part I. Accommodation of intermittent feeding and analysis of staged reactors. Biotechnology and Bioengineering 102:59-65.
South C R, Hogsett D A L, Lynd L R. 1995. Modeling simultaneous saccharification and fermentation of lignocellulose to ethanol in batch and continuous reactors. Enzyme and Microbial Technology 17:797-803.
Syamlal, M., O'Brien, T. J. 1988. Simulation of granular layer inversion in liquid fluidized beds. International Journal of Multiphase Flow. 14(4):473-481.
White F. 1991. Viscous fluid flow. McGraw-Hill International Editions. Singapore.
Zhang J, Shao X, Townsend O V, Lynd L R. 2009. Simultaneous saccharification and co-fermentation of paper sludge to ethanol by Saccharomyces cerevisiae RWB222-Part 1: Kinetic modeling and parameters. Biotechnology and Bioengineering. 104:920-931.

1. A computer-implemented method for controlling the conversion of crystalline insoluble cellulose to an organic product in a bioreactor containing crystalline insoluble cellulose and a culture medium, the method conducted at a processor of a computing device associated with the bioreactor and comprising:
receiving an input from a sensor in the bioreactor, wherein the input is measurements of one or more of concentration, temperature, pH, and pressure;
calculating conversion of cellulose using the input to provide a total calculated organic product in the bioreactor by solving the following equations:
$$\begin{matrix}\frac{d [ EC ]}{d t} = \frac{d [ C ]}{d t} ( 1 + {σ}_{e} ) + {k}_{fc} [ {E}_{f} ] [ {C}_{f} ] ( 1 + {σ}_{e} ) - \frac{{k}_{fc}}{K} [ EC ] & ( 1 ) \\ [ {E}_{f} ] = [ {E}_{T} ] - \frac{[ EC ] \times σ}{( 1 + σ )} & ( 2 ) \\ [ {C}_{f} ] = [ {C}_{T} ] - \frac{[ EC ]}{( 1 + σ )} & ( 3 ) \\ \frac{d [ C ]}{d t} = - k ( \frac{[ EC ]}{1 + σ} ) \times ( \frac{{K}_{C\_Cb}}{[ Cb ] + {K}_{C\_Cb}} ) \times ( \frac{{K}_{C\_Op}}{[ Op ] + {K}_{C\_Op}} ) & ( 4 ) \\ \frac{d [ Cb ]}{d t} = {K}_{f 1} \frac{d [ C ]}{d t} - \frac{{K}_{Cb} [ Cb ] [ B ]}{{K}_{m} \times ( ( 1 + \frac{[ G ]}{{K}_{Cb\_G}} ) + [ Cb ] )} & ( 5 ) \\ \frac{d [ G ]}{d t} = ( {K}_{f 1} \frac{d [ C ]}{d t} - \frac{d [ Cb ]}{d t} ) {K}_{f 2} - \frac{1}{{Y}_{X\_G}} \frac{d [ X ]}{d t} & ( 6 ) \\ \frac{d [ X ]}{d t} = \frac{{μ}_{max} [ X ] [ G ]}{[ G ] + {K}_{G}} \times ( 1 - \frac{[ Op ]}{{K}_{X\_Op}} ) & ( 7 ) \\ \frac{d [ Op ]}{d t} = ( \frac{{Y}_{Op\_G}}{{Y}_{X\_G}} ) \times \frac{d [ X ]}{d t} & ( 8 )\end{matrix}$$
where:
K_{C}_{_}_{Cb}=Inhibition constant of cellobiose on cellulose conversion [g/L]
K_{C}_{_}_{Op}=Inhibition constant of organic product on cellulose conversion [g/L]
K_{Cb}=Rate constant for hydrolysis of cellobiose to glucose [g/L]
K_{Cb}_{_}_{G}=Inhibition of hydrolysis of cellobiose by glucose [g/L]
K=Equilibrium constant of enzyme [L/g]
k=Hydrolysis rate constant of enzyme [h⁻¹]
k_{fc}=Enzyme adsorption constant to cellulose [h⁻¹]
K_{G}=Monod constant [g/L]
Kₘ=Michaelis constant of enzyme for cellobiose [g/L]
K_{X}_{_}_{Op}=Inhibition of cell growth by organic product [g/L]
Y_{Op}_{_}_{G}=Yield of organic product cells per gram of glucose
Y_{X}_{_}_{G}=Yield of organism cells per gram of glucose
μₘₐₓ=Maximum growth rate of organism cells [h⁻¹]
σₑ=Maximum bonding capacity of enzyme [dimensionless]
receiving a further input from a sensor in the bioreactor of the total actual organic product;
comparing the total calculated organic product and the total actual organic product; and
transmitting an instruction to an agitator associated with the bioreactor to control agitation of the content of the bioreactor if the total actual organic product is outside a predetermined range of the total calculated organic product.

2. The method as claimed in claim 1 further comprising the processor receiving inputs from a plurality of sensors wherein the inputs are measurements of concentration and wherein the concentration is enzyme loading concentration, cellulose concentration and organism concentration.

3. The method as claimed in claim 1 further comprising the processor solving equations (1) to (8) iteratively.

4. The method as claimed in claim 1 further comprising the processor receiving an additional input from a sensor in the bioreactor of a measurement relating to the rate of formation of enzyme-substrate complexes and calculating conversion of cellulose using this additional input to provide the total calculated organic product.

5. The method as claimed in claim 1 further comprising the processor receiving an additional input from a sensor of a measurement relating to the oxygen supplied to the bioreactor, and calculating conversion of cellulose using this additional input to provide the total calculated organic product.

6. The method as claimed in claim 1 further comprising, if the total actual organic product is outside the predetermined range of the total calculated organic product, the processor transmitting an instruction to a heater associated with the bioreactor to control temperature in the bioreactor, transmitting an instruction to an inlet valve of the bioreactor to control addition of an acid or base to control pH in the bioreactor, and transmitting an instruction to a pressurizing component associated with the bioreactor to control pressure in the bioreactor.

7. The method as claimed in claim 6 further comprising the processor transmitting instructions to control one or more of temperature, pH, and pressure within predetermined ranges.

8. The method as claimed in claim 7 further comprising the processor transmitting an instruction to an outlet valve of the bioreactor to cause purging of cellulose from the bioreactor if temperature is outside a predetermined temperature range.

9. The method as claimed in claim 1 further comprising the processor receiving an input from a sensor relating to the degree of settling of particles in the medium in which the conversion of crystalline insoluble cellulose takes place, comparing the input to a predetermined settling threshold, and transmitting an instruction to an agitator associated with the bioreactor to control agitation of the content of the bioreactor if the comparison is outside the predetermined settling threshold.

10. The method as claimed in claim 1 wherein the organic product is ethanol and the culture medium includes Saccharomyces cerevisiae or Bakers' yeast and wherein the method comprises:
receiving the following inputs from one or more sensors in the bioreactor:
Yeast cell concentration [g/L]—([X])
Cellulose concentration [g/L]—([C])
Cellobiose concentration [g/L]—([Cb])
Exo-cellulase enzyme concentration [g/L]—([Eₑₓₒ])
Endo-cellulase enzyme concentration [g/L]—([E_{endo}])
β-Glucosidase concentration [g/L]—([B])
Cellulose-enzyme complex concentration [g/L]—([EC]ₑₓₒ),
Cellulose-enzyme complex concentration [g/L]—([EC]_{endo}),
Ethanol concentration [g/L]—([Eth])
Glucose concentration [g/L]—([G])
calculating conversion of cellulose using these inputs to provide a total calculated ethanol in the bioreactor by solving the following equations:
$$\begin{matrix}\frac{d {[ EC ]}_{endo}}{d t} = \frac{d {[ C ]}_{endo}}{d t} \times ( 1 + {σ}_{endo} ) + {k}_{fc} [ {E}_{f , endo} ] [ {C}_{f , endo} ] ( 1 + {σ}_{endo} ) - {\frac{{k}_{fc}}{{K}_{endo}} [ EC ]}_{endo} & ( 9 ) \\ \frac{d {[ EC ]}_{exo}}{d t} = \frac{d {[ C ]}_{exo}}{d t} \times ( 1 + {σ}_{exo} ) + {k}_{fc} [ {E}_{f , exo} ] [ {C}_{f , exo} ] ( 1 + {σ}_{exo} ) - {\frac{{k}_{fc}}{{K}_{exo}} [ EC ]}_{exo} & ( 10 ) \\ [ {E}_{f} ] = [ {E}_{T} ] - \frac{[ EC ] \times σ}{( 1 + σ )} & ( 11 ) \\ [ {C}_{f} ] = [ {C}_{T} ] - \frac{[ EC ]}{( 1 + σ )} & ( 12 ) \\ \frac{d {[ C ]}_{endo}}{d t} = - {k}_{endo} \times \frac{{[ EC ]}_{endo}}{1 + {σ}_{endo}} \times ( \frac{{K}_{C\_Cb}}{[ Cb ] + {K}_{C\_Cb}} ) \times ( \frac{{K}_{C\_Eth}}{[ Eth ] + {K}_{C\_Eth}} ) & ( 13 ) \\ \frac{d {[ C ]}_{exo}}{d t} = tan h ( \frac{t}{τ} ) \times - {k}_{exo} \times \frac{{[ EC ]}_{exo}}{1 + {σ}_{exo}} \times ( \frac{{K}_{C\_Cb}}{[ Cb ] + {K}_{C\_Cb}} ) \times ( \frac{{K}_{C\_Eth}}{[ Eth ] + {K}_{C\_Eth}} ) & ( 14 ) \\ \frac{d [ Cb ]}{d t} = - \frac{342}{324} \times \frac{d [ C ]}{d t} - \frac{{K}_{Cb} [ Cb ] [ B ]}{{K}_{m} \times ( ( 1 + \frac{[ G ]}{{K}_{Cb\_G}} ) + [ Cb ] )} & ( 15 ) \\ \frac{d [ G ]}{d t} = ( - \frac{342}{324} \times \frac{d [ C ]}{d t} - \frac{d [ Cb ]}{d t} ) \times \frac{360}{342} - \frac{1}{{Y}_{X\_G}} \times \frac{d [ X ]}{d t} & ( 16 ) \\ \frac{d [ X ]}{d t} = \frac{{μ}_{max} [ X ] [ G ]}{[ G ] + {K}_{G}} \times ( 1 - \frac{[ Eth ]}{{K}_{X\_Eth}} ) & ( 17 ) \\ \frac{d [ Eth ]}{d t} = ( \frac{{Y}_{Eth\_G}}{{Y}_{X\_G}} ) \times \frac{d [ X ]}{d t} & ( 18 )\end{matrix}$$
where:
K_{C}_{_}_{Cb}=Inhibition constant of cellobiose on cellulose conversion [g/L]
K_{C}_{_}_{Eth}=Inhibition constant of ethanol on cellulose conversion [g/L]
K_{Cb}=Rate constant for hydrolysis of cellobiose to glucose [g/L]
K_{Cb}_{_}_{G}=Inhibition of hydrolysis of cellobiose by glucose [g/L]
K_{endo}=Equilibrium constant for endoglucanase [L/g]
k_{endo}=Hydrolysis rate constant of endoglucanase [h⁻¹]
Kₑₓₒ=Equilibrium constant for exoglucanase [L/g]
kₑₓₒ=Hydrolysis rate constant of exoglucanase [h⁻¹]
k_{fc}=Enzyme adsorption constant to Cellulose [h⁻¹]
K_{G}=Monod constant [g/L]
Kₘ=Michaelis constant of β-glucosidase for cellobiose [g/L]
K_{X}_{_}_{Eth}=Inhibition of cell growth by ethanol [g/L]
Y_{Eth}_{_}_{G}=Yield of ethanol cells per gram of glucose
Y_{X}_{_}_{G}=Yield of yeast cells per gram of glucose
μₘₐₓ=Maximum growth rate of yeast cells [h⁻¹]
σ_{endo}=Endoglucanse enzyme capacity on cellulose [dimensionless]
σₑₓₒ=Exoglucanase enzyme capacity on cellulose [dimensionless]
τ=Time Constant [h]
receiving a further input from a sensor in the bioreactor of the total actual ethanol,
comparing the total calculated ethanol and the total actual ethanol; and
transmitting an instruction to an agitator associated with the bioreactor to control agitation of the content of the bioreactor if the total actual ethanol is outside a predetermined range of the total calculated ethanol.

11. The method as claimed in claim 10 further comprising the processor solving equations (9) to (18) iteratively.

12. The method as claimed in claim 1 wherein the organic product is glycerol and the culture medium includes Saccharomyces cerevisiae or Bakers' yeast and wherein the method comprises:
receiving the following inputs from one or more sensors in the bioreactor:
Yeast cell concentration [g/L]—([X])
Cellulose concentration [g/L]—([C])
Cellobiose concentration [g/L]—([Cb])
Exo-cellulase enzyme concentration [g/L]—([Eₑₓₒ])
Endo-cellulase enzyme concentration [g/L]—([E_{endo}])
β-Glucosidase concentration [g/L]—([B])
Cellulose-enzyme complex concentration [g/L]—([EC]ₑₓₒ),
Cellulose-enzyme complex concentration [g/L]—([EC]_{endo}),
Glycerol concentration [g/L]—([Gly])
Glucose concentration [g/L]—([G])
calculating conversion of cellulose using these inputs to provide a total calculated glycerol in the bioreactor by solving the following equations:
$$\begin{matrix}\frac{d {[ EC ]}_{endo}}{d t} = \frac{d {[ C ]}_{endo}}{d t} \times ( 1 + {σ}_{endo} ) + {k}_{fc} [ {E}_{f , endo} ] [ {C}_{f , endo} ] ( 1 + {σ}_{endo} ) - {\frac{{k}_{fc}}{{K}_{endo}} [ EC ]}_{endo} & ( 9 ) \\ \frac{d {[ EC ]}_{exo}}{d t} = \frac{d {[ C ]}_{exo}}{d t} \times ( 1 + {σ}_{exo} ) + {k}_{fc} [ {E}_{f , exo} ] [ {C}_{f , exo} ] ( 1 + {σ}_{exo} ) - {\frac{{k}_{fc}}{{K}_{exo}} [ EC ]}_{exo} & ( 10 ) \\ [ {E}_{f} ] = [ {E}_{T} ] - \frac{[ EC ] \times σ}{( 1 + σ )} & ( 11 ) \\ [ {C}_{f} ] = [ {C}_{T} ] - \frac{[ EC ]}{( 1 + σ )} & ( 12 ) \\ \frac{d {[ C ]}_{endo}}{d t} = - {k}_{endo} \times \frac{{[ EC ]}_{endo}}{1 + {σ}_{endo}} \times ( \frac{{K}_{C\_Cb}}{[ Cb ] + {K}_{C\_Cb}} ) \times ( \frac{{K}_{C\_Gly}}{[ Gly ] + {K}_{C\_Gly}} ) & ( 19 ) \\ \frac{d {[ C ]}_{exo}}{d t} = tan h ( \frac{t}{τ} ) \times - {k}_{exo} \times \frac{{[ EC ]}_{exo}}{1 + {σ}_{exo}} \times ( \frac{{K}_{C\_Cb}}{[ Cb ] + {K}_{C\_Cb}} ) \times ( \frac{{K}_{C\_Gly}}{[ Gly ] + {K}_{C\_Gly}} ) & ( 20 ) \\ \frac{d [ Cb ]}{d t} = - \frac{342}{324} \times \frac{d [ C ]}{d t} - \frac{{K}_{Cb} [ Cb ] [ B ]}{{K}_{m} \times ( ( 1 + \frac{[ G ]}{{K}_{Cb\_G}} ) + [ Cb ] )} & ( 15 ) \\ \frac{d [ G ]}{d t} = ( - \frac{342}{324} \times \frac{d [ C ]}{d t} - \frac{d [ Cb ]}{d t} ) \times \frac{360}{342} - \frac{1}{{Y}_{X\_G}} \times \frac{d [ X ]}{d t} & ( 16 ) \\ \frac{d [ X ]}{d t} = \frac{{μ}_{max} [ X ] [ G ]}{[ G ] + {K}_{G}} \times ( 1 - \frac{[ Gly ]}{{K}_{X\_Gly}} ) & ( 21 ) \\ \frac{d [ Gly ]}{d t} = ( \frac{{Y}_{Gly\_G}}{{Y}_{X\_G}} ) \times \frac{d [ X ]}{d t} & ( 22 )\end{matrix}$$
where:
K_{C}_{_}_{Gly}=Inhibition constant of glycerol on cellulose conversion [g/L]
K_{X}_{_}_{Gly}=Inhibition of cell growth by glycerol [g/L]
Y_{Gly}_{_}_{G}=Yield of glycerol cells per gram of glucose
receiving a further input from a sensor in the bioreactor of the total actual glycerol,
comparing the total calculated glycerol and the total actual glycerol; and
transmitting an instruction to an agitator associated with the bioreactor to control agitation of the content of the bioreactor if the total actual glycerol is outside a predetermined range of the total calculated glycerol.

13. The method as claimed in claim 12 further comprising the processor solving equations (9) to (12), (15) to (16) and (19) to (22) iteratively.

14. A system for controlling the conversion of crystalline insoluble cellulose to an organic product in a bioreactor which can hold crystalline insoluble cellulose and a culture medium, the system comprising a computing device with memory for storing computer-readable program code and a processor for executing the computer-readable program code, wherein the processor is configured to interact with one or more sensors in the bioreactor, and an agitator associated with the bioreactor, and wherein the processor comprises:
a receiving component for receiving an input from a sensor, wherein the input is measurements of one or more of concentration, temperature, pH and pressure;
a calculating component for calculating conversion of cellulose using the input to provide a total calculated organic product in the bioreactor by solving the following equations:
$$\begin{matrix}\frac{d [ EC ]}{d t} = \frac{d [ C ]}{d t} ( 1 + {σ}_{e} ) + {k}_{fc} [ {E}_{f} ] [ {C}_{f} ] ( 1 + {σ}_{e} ) - \frac{{k}_{fc}}{K} [ EC ] & ( 1 ) \\ [ {E}_{f} ] = [ {E}_{T} ] - \frac{[ EC ] \times σ}{( 1 + σ )} & ( 2 ) \\ [ {C}_{f} ] = [ {C}_{T} ] - \frac{[ EC ]}{( 1 + σ )} & ( 3 ) \\ \frac{d [ C ]}{d t} = - k ( \frac{[ EC ]}{1 + σ} ) \times ( \frac{{K}_{C\_Cb}}{[ Cb ] + {K}_{C\_Cb}} ) \times ( \frac{{K}_{C\_Op}}{[ Op ] + {K}_{C\_Op}} ) & ( 4 ) \\ \frac{d [ Cb ]}{d t} = {K}_{f 1} \frac{d [ C ]}{d t} - \frac{{K}_{Cb} [ Cb ] [ B ]}{{K}_{m} \times ( ( 1 + \frac{[ G ]}{{K}_{Cb\_G}} ) + [ Cb ] )} & ( 5 ) \\ \frac{d [ G ]}{d t} = ( {K}_{f 1} \frac{d [ C ]}{d t} - \frac{d [ Cb ]}{d t} ) {K}_{f 2} - \frac{1}{{Y}_{X\_G}} \frac{d [ X ]}{d t} & ( 6 ) \\ \frac{d [ X ]}{d t} = \frac{{μ}_{max} [ X ] [ G ]}{[ G ] + {K}_{G}} \times ( 1 - \frac{[ Op ]}{{K}_{X\_Op}} ) & ( 7 ) \\ \frac{d [ Op ]}{d t} = ( \frac{{Y}_{Op\_G}}{{Y}_{X\_G}} ) \times \frac{d [ X ]}{d t} & ( 8 )\end{matrix}$$
where:
K_{C}_{_}_{Cb}=Inhibition constant of cellobiose on cellulose conversion [g/L]
K_{C}_{_}_{Op}=Inhibition constant of organic product on cellulose conversion [g/L]
K_{Cb}=Rate constant for hydrolysis of cellobiose to glucose [g/L]
K_{Cb}_{_}_{G}=Inhibition of hydrolysis of cellobiose by glucose [g/L]
K=Equilibrium constant of enzyme [L/g]
k=Hydrolysis rate constant of enzyme [h⁻¹]
k_{fc}=Enzyme adsorption constant to cellulose [h⁻¹]
K_{G}=Monod constant [g/L]
Kₘ=Michaelis constant of enzyme for cellobiose [g/L]
K_{X}_{_}_{Op}=Inhibition of cell growth by organic product [g/L]
Y_{Op}_{_}_{G}=Yield of organic product cells per gram of glucose
Y_{X}_{_}_{G}=Yield of organism cells per gram of glucose
μₘₐₓ=Maximum growth rate of organism cells [h⁻¹]
σₑ=Maximum bonding capacity of enzyme [dimensionless]
the receiving component receiving a further input from a sensor in the bioreactor of the total actual organic product;
a comparing component for comparing the total calculated organic product and the total actual organic product; and
an agitating component for transmitting an instruction to an agitator associated with the bioreactor to control agitation of the content of the bioreactor if the total actual organic product is outside a predetermined range of the total calculated organic product.

15. The system as claimed in claim 14 wherein the processor includes a temperature component for transmitting an instruction to a heater associated with the bioreactor to control temperature in the bioreactor, a pH component for transmitting an instruction to an inlet valve of the bioreactor to control addition of an acid or base to control pH in the bioreactor and a pressure component for transmitting an instruction to a pressurizing component associated with the bioreactor to control pressure in the bioreactor, if the total actual organic product is outside the predetermined range of the total calculated organic product.

16. The method as claimed in claim 15 wherein the temperature component, pH component and pressure component are configured to transmit instructions to control temperature, pH, and pressure within predetermined ranges.

17. The method as claimed in claim 14 wherein the receiving component of the processor is further configured to receive input from a sensor relating to the degree of settling of particles in the medium in which the conversion of crystalline insoluble cellulose takes place, the comparing component of the processor is configured to compare the input to a predetermined settling threshold, and the agitating component of the processor is configured to transmit an instruction to an agitator associated with the bioreactor to control agitation of the content of the bioreactor if the comparison is outside the predetermined settling threshold.

18. The system as claimed in claim 14 wherein the organic product is ethanol and the culture medium includes Saccharomyces cerevisiae and wherein the system comprises:
the receiving component receiving the following inputs from one or more sensors in the bioreactor:
Yeast cell concentration [g/L]—([X])
Cellulose concentration [g/L]—([C])
Cellobiose concentration [g/L]—([Cb])
Exo-cellulase enzyme concentration [g/L]—([Eₑₓₒ])
Endo-cellulase enzyme concentration [g/L]—([E_{endo}])
β-Glucosidase concentration [g/L]—([B])
Cellulose-enzyme complex concentration [g/L]—([EC]ₑₓₒ),
Cellulose-enzyme complex concentration [g/L]—([EC]_{endo}),
Ethanol concentration [g/L]—([Eth])
Glucose concentration [g/L]—([G])
the calculating component calculating conversion of cellulose using these inputs to provide a total calculated ethanol in the bioreactor by solving the following equations:
$$\begin{matrix}\frac{d {[ EC ]}_{endo}}{d t} = \frac{d {[ C ]}_{endo}}{d t} \times ( 1 + {σ}_{endo} ) + {k}_{fc} [ {E}_{f , endo} ] [ {C}_{f , endo} ] ( 1 + {σ}_{endo} ) - {\frac{{k}_{fc}}{{K}_{endo}} [ EC ]}_{endo} & ( 9 ) \\ \frac{d {[ EC ]}_{exo}}{d t} = \frac{d {[ C ]}_{exo}}{d t} \times ( 1 + {σ}_{exo} ) + {k}_{fc} [ {E}_{f , exo} ] [ {C}_{f , exo} ] ( 1 + {σ}_{exo} ) - {\frac{{k}_{fc}}{{K}_{exo}} [ EC ]}_{exo} & ( 10 ) \\ [ {E}_{f} ] = [ {E}_{T} ] - \frac{[ EC ] \times σ}{( 1 + σ )} & ( 11 ) \\ [ {C}_{f} ] = [ {C}_{T} ] - \frac{[ EC ]}{( 1 + σ )} & ( 12 ) \\ \frac{d {[ C ]}_{endo}}{d t} = - {k}_{endo} \times \frac{{[ EC ]}_{endo}}{1 + {σ}_{endo}} \times ( \frac{{K}_{C\_Cb}}{[ Cb ] + {K}_{C\_Cb}} ) \times ( \frac{{K}_{C\_Eth}}{[ Eth ] + {K}_{C\_Eth}} ) & ( 13 ) \\ \frac{d {[ C ]}_{exo}}{d t} = tan h ( \frac{t}{τ} ) \times - {k}_{exo} \times \frac{{[ EC ]}_{exo}}{1 + {σ}_{exo}} \times ( \frac{{K}_{C\_Cb}}{[ Cb ] + {K}_{C\_Cb}} ) \times ( \frac{{K}_{C\_Eth}}{[ Eth ] + {K}_{C\_Eth}} ) & ( 14 ) \\ \frac{d [ Cb ]}{d t} = - \frac{342}{324} \times \frac{d [ C ]}{d t} - \frac{{K}_{Cb} [ Cb ] [ B ]}{{K}_{m} \times ( ( 1 + \frac{[ G ]}{{K}_{Cb\_G}} ) + [ Cb ] )} & ( 15 ) \\ \frac{d [ G ]}{d t} = ( - \frac{342}{324} \times \frac{d [ C ]}{d t} - \frac{d [ Cb ]}{d t} ) \times \frac{360}{342} - \frac{1}{{Y}_{X\_G}} \times \frac{d [ X ]}{d t} & ( 16 ) \\ \frac{d [ X ]}{d t} = \frac{{μ}_{max} [ X ] [ G ]}{[ G ] + {K}_{G}} \times ( 1 - \frac{[ Eth ]}{{K}_{X\_Eth}} ) & ( 17 ) \\ \frac{d [ Eth ]}{d t} = ( \frac{{Y}_{Eth\_G}}{{Y}_{X\_G}} ) \times \frac{d [ X ]}{d t} & ( 18 )\end{matrix}$$
where:
K_{C}_{_}_{Cb}=Inhibition constant of cellobiose on cellulose conversion [g/L]
K_{C}_{_}_{Eth}=Inhibition constant of ethanol on cellulose conversion [g/L]
K_{Cb}=Rate constant for hydrolysis of cellobiose to glucose [g/L]
K_{Cb}_{_}_{G}=Inhibition of hydrolysis of cellobiose by glucose [g/L]
K_{endo}=Equilibrium constant for endoglucanase [L/g]
k_{endo}=Hydrolysis rate constant of endoglucanase [h⁻¹]
Kₑₓₒ=Equilibrium constant for exoglucanase [L/g]
kₑₓₒ=Hydrolysis rate constant of exoglucanase [h⁻¹]
k_{fc}=Enzyme adsorption constant to Avicel [h⁻¹]
K_{G}=Monod constant [g/L]
Kₘ=Michaelis constant of β-glucosidase for cellobiose [g/L]
K_{X}_{_}_{Eth}=Inhibition of cell growth by ethanol [g/L]
Y_{Eth}_{_}_{G}=Yield of ethanol cells per gram of glucose
Y_{X}_{_}_{G}=Yield of yeast cells per gram of glucose
μₘₐₓ=Maximum growth rate of yeast cells [h⁻¹]
σ_{endo}=Endoglucanse enzyme capacity on Avicel [dimensionless]
σₑₓₒ=Exoglucanase enzyme capacity on Avicel [dimensionless]
τ=Time Constant [h]
the receiving component receiving a further input from a sensor in the bioreactor of the total actual ethanol;
the comparing component comparing the total calculated ethanol and the total actual ethanol; and
the agitating component transmitting an instruction to an agitator associated with the bioreactor to control agitation of the content of the bioreactor if the total actual ethanol is outside a predetermined range of the total calculated ethanol.

19. The system as claimed in claim 14 the organic product is glycerol and the culture medium includes Saccharomyces cerevisiae and wherein the system comprises:
the receiving component receiving the following inputs from one or more sensors in the bioreactor:
Yeast cell concentration [g/L]—([X])
Cellulose concentration [g/L]—([C])
Cellobiose concentration [g/L]—([Cb])
Exo-cellulase enzyme concentration [g/L]—([Eₑₓₒ])
Endo-cellulase enzyme concentration [g/L]—([E_{endo}])
β-Glucosidase concentration [g/L]—([B])
Cellulose-enzyme complex concentration [g/L]—([EC]ₑₓₒ),
Cellulose-enzyme complex concentration [g/L]—([EC]_{endo}),
Glycerol concentration [g/L]—([Gly])
Glucose concentration [g/L]—([G])
the calculating component calculating conversion of cellulose using these inputs to provide a total calculated glycerol in the bioreactor by solving the following equations:
$$\begin{matrix}\frac{d {[ EC ]}_{endo}}{d t} = \frac{d {[ C ]}_{endo}}{d t} \times ( 1 + {σ}_{endo} ) + {k}_{fc} [ {E}_{f , endo} ] [ {C}_{f , endo} ] ( 1 + {σ}_{endo} ) - {\frac{{k}_{fc}}{{K}_{endo}} [ EC ]}_{endo} & ( 9 ) \\ \frac{d {[ EC ]}_{exo}}{d t} = \frac{d {[ C ]}_{exo}}{d t} \times ( 1 + {σ}_{exo} ) + {k}_{fc} [ {E}_{f , exo} ] [ {C}_{f , exo} ] ( 1 + {σ}_{exo} ) - {\frac{{k}_{fc}}{{K}_{exo}} [ EC ]}_{exo} & ( 10 ) \\ [ {E}_{f} ] = [ {E}_{T} ] - \frac{[ EC ] \times σ}{( 1 + σ )} & ( 11 ) \\ [ {C}_{f} ] = [ {C}_{T} ] - \frac{[ EC ]}{( 1 + σ )} & ( 12 ) \\ \frac{d {[ C ]}_{endo}}{d t} = - {k}_{endo} \times \frac{{[ EC ]}_{endo}}{1 + {σ}_{endo}} \times ( \frac{{K}_{C\_Cb}}{[ Cb ] + {K}_{C\_Cb}} ) \times ( \frac{{K}_{C\_Gly}}{[ Gly ] + {K}_{C\_Gly}} ) & ( 19 ) \\ \frac{d {[ C ]}_{exo}}{d t} = tan h ( \frac{t}{τ} ) \times - {k}_{exo} \times \frac{{[ EC ]}_{exo}}{1 + {σ}_{exo}} \times ( \frac{{K}_{C\_Cb}}{[ Cb ] + {K}_{C\_Cb}} ) \times ( \frac{{K}_{C\_Gly}}{[ Gly ] + {K}_{C\_Gly}} ) & ( 20 ) \\ \frac{d [ Cb ]}{d t} = - \frac{342}{324} \times \frac{d [ C ]}{d t} - \frac{{K}_{Cb} [ Cb ] [ B ]}{{K}_{m} \times ( ( 1 + \frac{[ G ]}{{K}_{Cb\_G}} ) + [ Cb ] )} & ( 15 ) \\ \frac{d [ G ]}{d t} = ( - \frac{342}{324} \times \frac{d [ C ]}{d t} - \frac{d [ Cb ]}{d t} ) \times \frac{360}{342} - \frac{1}{{Y}_{X\_G}} \times \frac{d [ X ]}{d t} & ( 16 ) \\ \frac{d [ X ]}{d t} = \frac{{μ}_{max} [ X ] [ G ]}{[ G ] + {K}_{G}} \times ( 1 - \frac{[ Gly ]}{{K}_{X\_Gly}} ) & ( 21 ) \\ \frac{d [ Gly ]}{d t} = ( \frac{{Y}_{Gly\_G}}{{Y}_{X\_G}} ) \times \frac{d [ X ]}{d t} & ( 22 )\end{matrix}$$
where:
K_{C}_{_}_{Gly}=Inhibition constant of glycerol on cellulose conversion [g/L]
K_{X}_{_}_{Gly}=Inhibition of cell growth by glycerol [g/L]
Y_{Gly}_{_}_{G}=Yield of glycerol cells per gram of glucose
the receiving component receiving a further input from a sensor in the bioreactor of the total actual glycerol;
the comparing component comparing the total calculated glycerol and the total actual glycerol; and
the agitating component transmitting an instruction to an agitator associated with the bioreactor to control agitation of the content of the bioreactor if the total actual ethanol is outside a predetermined range of the total calculated glycerol.

20. A non-transitory computer program product for controlling the conversion of crystalline insoluble cellulose to an organic product in a bioreactor containing crystalline insoluble cellulose and a culture medium, the computer program product comprising a computer-readable medium having stored computer-readable program code for performing the steps of:
receiving an input from a sensor in the bioreactor, wherein the input is measurements of one or more of concentration, temperature, pH and pressure;
calculating conversion of cellulose using the input to provide a total calculated organic product in the bioreactor by solving the following equations:
$$\begin{matrix}\frac{d [ EC ]}{d t} = \frac{d [ C ]}{d t} ( 1 + {σ}_{e} ) + {k}_{fc} [ {E}_{f} ] [ {C}_{f} ] ( 1 + {σ}_{e} ) - \frac{{k}_{fc}}{K} [ EC ] & ( 1 ) \\ [ {E}_{f} ] = [ {E}_{T} ] - \frac{[ EC ] \times σ}{( 1 + σ )} & ( 2 ) \\ [ {C}_{f} ] = [ {C}_{T} ] - \frac{[ EC ]}{( 1 + σ )} & ( 3 ) \\ \frac{d [ C ]}{d t} = - k ( \frac{[ EC ]}{1 + σ} ) \times ( \frac{{K}_{C\_Cb}}{[ Cb ] + {K}_{C\_Cb}} ) \times ( \frac{{K}_{C\_Op}}{[ Op ] + {K}_{C\_Op}} ) & ( 4 ) \\ \frac{d [ Cb ]}{d t} = {K}_{f 1} \frac{d [ C ]}{d t} - \frac{{K}_{Cb} [ Cb ] [ B ]}{{K}_{m} \times ( ( 1 + \frac{[ G ]}{{K}_{Cb\_G}} ) + [ Cb ] )} & ( 5 ) \\ \frac{d [ G ]}{d t} = ( {K}_{f 1} \frac{d [ C ]}{d t} - \frac{d [ Cb ]}{d t} ) {K}_{f 2} - \frac{1}{{Y}_{X\_G}} \frac{d [ X ]}{d t} & ( 6 ) \\ \frac{d [ X ]}{d t} = \frac{{μ}_{max} [ X ] [ G ]}{[ G ] + {K}_{G}} \times ( 1 - \frac{[ Op ]}{{K}_{X\_Op}} ) & ( 7 ) \\ \frac{d [ Op ]}{d t} = ( \frac{{Y}_{Op\_G}}{{Y}_{X\_G}} ) \times \frac{d [ X ]}{d t} & ( 8 )\end{matrix}$$
where:
K_{C}_{_}_{Cb}=Inhibition constant of cellobiose on cellulose conversion [g/L]
K_{C}_{_}_{Op}=Inhibition constant of organic product on cellulose conversion [g/L]
K_{Cb}=Rate constant for hydrolysis of cellobiose to glucose [g/L]
K_{Cb}_{_}_{G}=Inhibition of hydrolysis of cellobiose by glucose [g/L]
K=Equilibrium constant of enzyme [L/g]
k=Hydrolysis rate constant of enzyme [h⁻¹]
k_{fc}=Enzyme adsorption constant to cellulose [h⁻¹]
K_{G}=Monod constant [g/L]
Kₘ=Michaelis constant of enzyme for cellobiose [g/L]
K_{X}_{_}_{Op}=Inhibition of cell growth by organic product [g/L]
Y_{Op}_{_}_{G}=Yield of organic product cells per gram of glucose
Y_{X}_{_}_{G}=Yield of organism cells per gram of glucose
μₘₐₓ=Maximum growth rate of organism cells [h⁻¹]
σₑ=Maximum bonding capacity of enzyme [dimensionless]
receiving a further input from a sensor in the bioreactor of the total actual organic product;
comparing the total calculated organic product and the total actual organic product; and
transmitting an instruction to an agitator associated with the bioreactor to control agitation of the content of the bioreactor if the total actual organic product is outside a predetermined range of the total calculated organic product.
